(19) United States
(12) Patent Application Publication
(10) Pub. No.: US 2022/0136068 A1
(43) Pub. Date: May 5, 2022

(54) METHODS OF DETECTING AND TREATING VENETOCLAX-RESISTANT ACUTE MYELOID LEUKEMIA

(71) Applicant: The Regents of the University of Colorado, A Body Corporate, Denver, CO (US)

(72) Inventors: Clayton SMITH, Aurora, CO (US); Craig T. JORDAN, Aurora, CO (US); Daniel POLLYEA, Aurora, CO (US); Shanshan PEI, Aurora, CO (US)

(21) Appl. No.: 17/435,728

(22) PCT Filed: Mar. 6, 2020
(86) PCT No.: PCT/US2020/021464

§ 371 Date: Sep. 2, 2021

Related U.S. Application Data
(60) Provisional application No. 62/887,982, filed on Aug. 16, 2019.
(60) Provisional application No. 62/814,711, filed on Mar. 6, 2019.

(51) Int. Cl. C12Q 1/6886; A61K 31/496; A61K 31/706; A61P 35/02

(52) U.S. Cl. CPC C12Q 1/6886; A61K 31/496; C12Q 2600/158; A61P 35/02; C12Q 2600/106; A61K 31/706

(57) ABSTRACT

The present disclosure relates to methods of identifying AML patients who will be resistant to venetoclax therapy and methods of treating venetoclax-resistant patients with myeloid leukemia cell differentiation protein (MCL-1) inhibitors.

### CROSS-REFERENCE TO RELATED APPLICATIONS

This application claims priority to, and the benefit of, U.S. Provisional Application No. 62/814,711 filed on Mar. 6, 2019 and U.S. Provisional Application No. 62/887,982 filed on Aug. 16, 2019. The contents of each of the aforementioned patent applications are incorporated herein by reference in their entireties.

### GOVERNMENT SUPPORT

This invention was made with government support under grant number R01CA200707 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 28, 2020, is named “UNCO-025_001WO_SeqList.txt” and is about 55.6 KB in size.

### BACKGROUND OF THE INVENTION

Acute myeloid leukemia (AML) is a blood cancer in which the bone marrow of a subject makes abnormal myeloblasts, red blood cells, or platelets. AML is one of the most common forms of acute leukemia in adults. The build-up of AML cells in bone marrow and blood can rapidly lead to infection, anemia, excessive bleeding and death. BCL-2 inhibitor venetoclax has recently emerged as an important component of therapy for acute myeloid leukemia (AML). In combination with a number of backbone chemotherapy treatments, venetoclax can induce responses in over 70% of older previously untreated AML patients, many of whom are unfit for conventional induction therapy. However, resistance to venetoclax-based therapy has been documented, as well as relapse following initial response. There is a need in the art for methods of predicting response to venetoclax treatment and methods of treating AML in patients who are predicted to be refractory to or to relapse after treatment with venetoclax. There is also a need in the art for methods of treating AML, particularly in elderly patients that are unfit for conventional induction therapy and for patients with relapsed AML.

### SUMMARY OF THE INVENTION

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be refractory to treatment with a combination of venetoclax and azacitidine when:

i) the sample is classified as FAB-M5,
the clinical karyotype is complex,
the side scatter intensity is greater than or equal to the first side scatter intensity predetermined cutoff value,
the expression level of each of CD11b and CD64 is greater than or equal to its corresponding predetermined cutoff value,
the expression level of CD45 is greater than or equal to its corresponding first predetermined cutoff value, and
the expression level of CD117 is less than its corresponding predetermined cutoff value; or

ii) the sample is classified as FAB-M0, FAB-M1 or FAB-M2,
the clinical karyotype is complex,
the side scatter intensity is less than the second side scatter intensity predetermined cutoff value,
the expression level of CD11b or CD64 is greater than or equal to its corresponding predetermined cutoff value,
the expression level of CD45 is less than its corresponding first predetermined cutoff value and greater than or equal to its corresponding second predetermined cutoff value, and
the expression level of CD117 is greater than or equal to its corresponding predetermined cutoff value.

The present disclosure provides a method of identifying a subject having AML that will relapse after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be relapse after treatment with a combination of venetoclax and azacitidine when:
the sample is classified as FAB-M4,
the clinical karyotype is complex,
the side scatter intensity is greater than or equal to the first side scatter intensity predetermined cutoff value and less than the second side scatter intensity predetermined cutoff value,
the expression level of each of CD45, CD11b and CD64 is greater than or equal to its corresponding predetermined cutoff value, and
the expression level of CD117 is less than its corresponding predetermined cutoff value.

The preceding method can further comprise identifying that the subject having AML that will relapse after treatment with a combination of venetoclax and azacitidine will also initially exhibit complete remission, complete remission with incomplete blood count recovery, morphologic leukemia-free state or any combination thereof after treatment with a combination of venetoclax and azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be exhibit durable remission after treatment with a combination of venetoclax and azacitidine when:

i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or

ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value, and
the expression level of CD117 is less than its corresponding predetermined cutoff value;

iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or

iv) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value.

The present disclosure provides a method of identifying a subject having acute myeloid leukemia (AML) that will be refractory to or that will relapse from treatment with a combination of venetoclax and azacitidine, the method comprising: a) determining the expression level of CD7 in a sample from the subject; b) comparing the expression level of CD7 to a predetermined cutoff value; and c) identifying that the subject will be refractory to treatment with a combination of venetoclax and azacitidine when the expression level of CD7 is equal to or greater than the predetermined cutoff.

In some aspects, a sample can comprise acute myeloid leukemia cells. In some aspects, acute myeloid leukemia cells can comprise acute myeloid leukemia blast cells. In some aspects, acute myeloid leukemia cells can comprise leukemia stem cells. In some aspects, leukemia stem cells can comprise reactive oxygen species-low leukemia stem cells.

In some aspects, a sample can comprise blood, a bone marrow biopsy, a bone marrow aspirate, a biopsy of a chloroma, a tissue biopsy, cerebrospinal fluid or any combination thereof. A sample can be a bone marrow biopsy. A sample can be a bone marrow aspirate. A sample can be a biopsy of a chloroma. A sample can comprise cerebrospinal fluid.

A subject can have been previously diagnosed with acute myeloid leukemia. A subject can have been previously administered an initial therapy. A subject can have not responded to the initial therapy. An initial therapy can comprise administering to the subject a therapeutically effective amount of venetoclax in combination with a therapeutically effective amount of azacitidine. An initial therapy can comprise anti-cancer therapy, chemotherapy, targeted drug therapy, radiation therapy, immunotherapy, stem cell transplant or any combination thereof.

A subject can be at least 18 years of age, or at least 50 years of age, or at least 60 years of age, or at least 70 years of age, or at least 80 years of age.

Expression of a biomarker or CD7 can comprise PCR, high-throughput sequencing, next generation sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

A complex clinical karyotype can comprise the presence of at least 3 chromosomal aberrations. A normal clinical karyotype can comprise 46 XY or 46 XX.

Methods of the present disclosure can further comprise administering at least one therapeutically effective amount of at least one alternative therapy to the subject that is identified as a subject that will be refractory to or that will relapse from treatment with a combination of venetoclax and azacitidine, wherein the at least one alternative therapy does not comprise venetoclax in combination with azacitidine.

Methods of the present disclosure can further comprise providing a treatment recommendation to the subject that is identified as a subject that will be refractory to or that will relapse from treatment with a combination of venetoclax and azacitidine, wherein the treatment recommendation comprises recommending the administration of at least one therapeutically effective amount of at least one alternative therapy.

An at least one alternative therapy can comprise anti-cancer therapy, chemotherapy, targeted drug therapy, radiation therapy, immunotherapy, stem cell transplant or any combination thereof.

Methods of the present disclosure can further comprise administering at least one therapeutically effective amount of venetoclax in combination with a therapeutically effective amount of azacitidine to the subject that is identified as a subject that will exhibit durable remission after treatment with a combination of venetoclax and azacitidine.

Methods of the present disclosure can further comprise providing a treatment recommendation to the subject that is identified as a subject that will exhibit durable remission after treatment with a combination of venetoclax and azacitidine, wherein the treatment recommendation comprises recommending the administration of at least one therapeutically effective amount of venetoclax in combination with a therapeutically effective amount of azacitidine.

The present disclosure provides a method of treating acute myeloid leukemia in a subject comprising administering to the subject a therapeutically effective amount of at least one MCL-1 inhibitor in combination with a therapeutically effective amount of azacitidine.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Unless specifically stated or obvious from context, as used herein, the term “or” is understood to be inclusive and covers both “or” and “and”.

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 shows a time line and in vivo treatment plans for the VEN+AZA regimen used for the PDX experiment. OG=oral gavage, IP=intraperitoneal injection.

FIG. 2 is a series of charts showing viability of sorted ROSlow LSCs from mono-AML (n=5) and prim-AML (n=7) after 24 hours in vitro treatment with venetoclax alone or in combination with a fixed dose of 1.5 μM azacitidine. Each data point was expressed as mean±SD of technical triplicates. All viability data were normalized to untreated control.

FIG. 3 is a series of bar graphs viability of three mono-AMLs from the Research Cohort 2 after 24 hours in vitro treatment with various single or combinations drugs.

FIG. 4 is a series of bar graph showing viability of three mono-AMLs from the Trial Cohort 1 after 24 hours in vitro treatment with various single or combo drugs. Mono-AMLs from the Trial Cohort 1 were isolated from bone marrow specimens at refractory (Patient-72) or relapsed (Patient-12 and Patient-65) stages.

FIG. 5 is a series of bar graphs showing Colony Forming Unit (CFU) assay results comparing impacts of 0.5 μM venetoclax+1.5 μM AZA versus 0.5 μM VU103+1.5 μM AZA on the progenitor function of two mono-AMLs from the Research Cohort 2 and one mono-AML from the Trial Cohort 1.

FIG. 6 is a series of graphs showing the percentage of engraftment in NSG-S mice after ex vivo treatment with 0.5 μM venetoclax+1.5 μM azacitidine or 0.5 μM VU103+1.5 μM azacitidine. Each dot represents an individual mouse. Median+/−Interquartile. Mann-Whitney test was used to compare the treatment groups. *** and ** indicates significance with p value of less than 0.001 and 0.01, respectively.

FIG. 7 is a schematic illustration of the identified significant biological differences between primitive and monocytic AML and their implications for predicting resistance to venetoclax-based therapy and sensitivity to MCL-1 inhibitors.

FIG. 8 is a schematic overview of a prognosis algorithm of the present disclosure for AML patients treated with VEN+AZA.

FIG. 9 is a table showing the karyotype analysis of various patient samples and their FAB, refractory and relapse status.

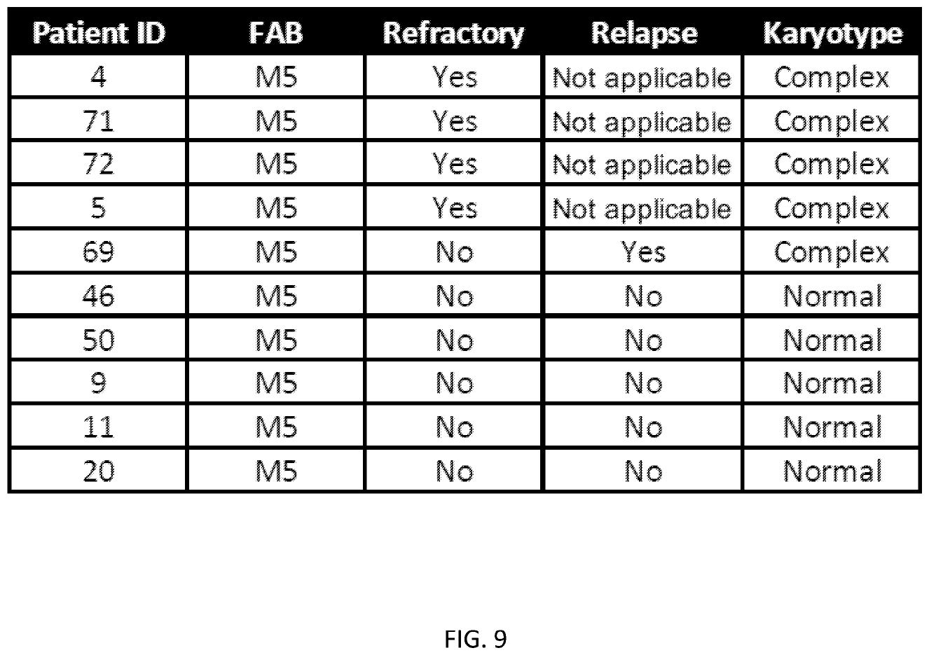

FIG. 10 is a graph showing the response to venetoclax and azacitidine combination treatment in patients with CD7− and CD7+ acute myeloid leukemia.

### DETAILED DESCRIPTION OF THE INVENTION

Acute myeloid leukemia is a blood cancer that is one of the most commonly diagnosed types of leukemia in adults. It is estimated that there will be approximately 21,000 new cases of acute myeloid leukemia diagnosed in the United States in 2019. The average age of a person diagnosed with acute myeloid leukemia is about 68, with most cases occurring after the age of 45. However, acute myeloid leukemia has also been diagnosed in younger patients, including children. Prognosis for patients diagnosed with acute myeloid leukemia is generally poor, with a long-term survival of only 40-50% in younger patients and a median overall survival of less than one year for older patients. New therapies aimed at supplementing the standard remission induction regimen of infusional cytarabine with intermittent dosing of an anthracycline have not yielded additional clinical benefits. Thus, there exists a need for more specialized and personalized treatment methods, particularly in older patients who are unfit for induction therapy.

As an alternative to standard induction therapy in elderly patients, the standard of care has been to offer low-dose therapies such as hypomethylating agents (HMA), which result in few responders and negligible long-term overall survival. Recent clinical trials have reported that the addition of the highly specific BCL-2 inhibitor venetoclax to the HMA backbone can greatly increase the response rates and potentially the overall survival (OS) for older, newly diagnosed AML patients who are unfit for conventional chemotherapy^{6,7}. This led to the recent United States Food and Drug Administration approval of this regimen for this population, and it is now considered to be the standard care.

The combination of venetoclax and the HMA azacitidine has resulted in a remission rate of over 70%. However, a significant minority of patients did not achieve a remission and were refractory. In addition, the majority of patients who did achieve a remission ultimately relapsed. Thus, there exists a need in the art for methods of predicting response to venetoclax in combination with azacitidine treatment and methods of treating AML in patients who are predicted to be refractory to or predicted to relapse after treatment with venetoclax in combination with azacitidine treatment.

Recent research has also demonstrated that acute myeloid leukemia exhibits a high level of biological heterogeneity, potentially explaining the difficulty in finding effective therapeutic strategies for the treatment of AML. Furthermore, it has been recently recognized that leukemia stem cells (LSCs), which are capable of giving rise to identical daughter cells as well as differentiated cells, perpetuate and maintain acute myeloid leukemia.

The present disclosure provides methods for the treatment and prognosis of acute myeloid leukemia in a subject. Various methods of the present disclosure are described in full detail herein.

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be refractory to treatment with a combination of venetoclax and azacitidine when:

i) the sample is classified as FAB-M5,
the clinical karyotype is complex,
the side scatter intensity is greater than or equal to the first side scatter intensity predetermined cutoff value,
the expression level of each of CD11b and CD64 is greater than or equal to its corresponding predetermined cutoff value,
the expression level of CD45 is greater than or equal to its corresponding first predetermined cutoff value, and
the expression level of CD117 is less than its corresponding predetermined cutoff value; or

ii) the sample is classified as FAB-M0, FAB-M1 or FAB-M2,
the clinical karyotype is complex,
the side scatter intensity is less than the second side scatter intensity predetermined cutoff value,
the expression level of CD11b or CD64 is greater than or equal to its corresponding predetermined cutoff value,
the expression level of CD45 is less than its corresponding first predetermined cutoff value and greater than or equal to its corresponding second predetermined cutoff value, and
the expression level of CD117 is greater than or equal to its corresponding predetermined cutoff value.

The preceding method can further comprise administering at least one therapy to the subject identified as being refractory to treatment with a combination of venetoclax and azacitidine, wherein the at least one therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) administering a first therapy to the subject when:

i) the sample is classified as FAB-M5,
the clinical karyotype is complex,
the side scatter intensity is greater than or equal to the first side scatter intensity predetermined cutoff value,
the expression level of each of CD11b and CD64 is greater than or equal to its corresponding predetermined cutoff value,
the expression level of CD45 is greater than or equal to its corresponding first predetermined cutoff value, and
the expression level of CD117 is less than its corresponding predetermined cutoff value; or

ii) the sample is classified as FAB-M0, FAB-M1 or FAB-M2,
the clinical karyotype is complex,
the side scatter intensity is less than the second side scatter intensity predetermined cutoff value,
the expression level of CD11b or CD64 is greater than or equal to its corresponding predetermined cutoff value,
the expression level of CD45 is less than its corresponding first predetermined cutoff value and greater than or equal to its corresponding second predetermined cutoff value, and
the expression level of CD117 is greater than or equal to its corresponding predetermined cutoff value,
   wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having AML that will relapse after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be relapse after treatment with a combination of venetoclax and azacitidine when:
the sample is classified as FAB-M4,
the clinical karyotype is complex,
the side scatter intensity is greater than or equal to the first side scatter intensity predetermined cutoff value and less than the second side scatter intensity predetermined cutoff value,
the expression level of each of CD45, CD11b and CD64 is greater than or equal to its corresponding predetermined cutoff value, and
the expression level of CD117 is less than its corresponding predetermined cutoff value.

The preceding method can further comprise administering at least one therapy to the subject identified as relapsing after treatment with a combination of venetoclax and azacitidine, wherein the at least one therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when:
the sample is classified as FAB-M4,
the clinical karyotype is complex,
the side scatter intensity is greater than or equal to the first side scatter intensity predetermined cutoff value and less than the second side scatter intensity predetermined cutoff value,
the expression level of each of CD45, CD11b and CD64 is greater than or equal to its corresponding predetermined cutoff value, and
the expression level of CD117 is less than its corresponding predetermined cutoff value,
   wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be exhibit durable remission after treatment with a combination of venetoclax and azacitidine when:

i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or

ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value, and
the expression level of CD117 is less than its corresponding predetermined cutoff value;

iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or

iv) the sample is classified as FAB-M0, FAB-M1 or FAB-M2,
the clinical karyotype is normal,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value.

The preceding method can further comprise administering at least one therapy to the subject identified as exhibiting durable remission after treatment with a combination of venetoclax and azacitidine, wherein the at least one therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) administering a first therapy to the subject when:

i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or

ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value, and
the expression level of CD117 is less than its corresponding predetermined cutoff value;

iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or

iv) the sample is classified as FAB-M0, FAB-M1 or FAB-M2,
the clinical karyotype is normal,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value,
   wherein the first therapy comprises administering to the subject a therapeutically effective amount of venetoclax in combination with a therapeutically effective amount of azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be exhibit durable remission after treatment with a combination of venetoclax and azacitidine when:

i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or

ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value such that the sample is partially positive for CD11b and CD64, and
the expression level of CD117 is less than its corresponding predetermined cutoff value;

iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or

iv) the sample is classified as FAB-M0, FAB-M1 or FAB-M2,
the clinical karyotype is normal,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value.

The preceding method can further comprise administering at least one therapy to the subject identified as exhibiting durable remission after treatment with a combination of venetoclax and azacitidine, wherein the at least one therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and second predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) administering a first therapy to the subject when:

i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or

ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value such that the sample is partially positive for CD11b and CD64, and
the expression level of CD117 is less than its corresponding predetermined cutoff value;

iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or

iv) the sample is classified as FAB-M0, FAB-M1 or FAB-M2,
the clinical karyotype is normal,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value,
   wherein the first therapy comprises administering to the subject a therapeutically effective amount of venetoclax in combination with a therapeutically effective amount of azacitidine.

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be refractory to treatment with a combination of venetoclax and azacitidine when the sample from the subject is classified as FAB-M5, has a complex clinical karyotype and is CD45^{Bright}/SSC^{High}/CD117−/CD11b+/CD64+.

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be refractory to treatment with a combination of venetoclax and azacitidine when: the sample is classified as FAB-M5, the clinical karyotype is complex, the side scatter intensity is greater than or equal to the side scatter intensity predetermined cutoff value, the expression level of each of CD11b and CD64 is greater than or equal to its corresponding predetermined cutoff value, the expression level of CD45 is greater than or equal to its corresponding predetermined cutoff value, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M5, has a complex clinical karyotype and is CD45^{Bright}/SSC^{High}/CD117−/CD11b+/CD64+, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M5, the clinical karyotype is complex, the side scatter intensity is greater than or equal to the side scatter intensity predetermined cutoff value, the expression level of each of CD11b and CD64 is greater than or equal to its corresponding predetermined cutoff value, the expression level of CD45 is greater than or equal to its corresponding predetermined cutoff value, and the expression level of CD117 is less than its corresponding predetermined cutoff value, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be refractory to treatment with a combination of venetoclax and azacitidine when the sample from the subject is classified as FAB-M0, FAB-M1 or FAB-M2, has a complex clinical karyotype and is CD45^{Med}/SSC^{low}/CD117+/CD11b+ or CD45^{Med}/SSC^{low}/CD117+/CD64+.

The present disclosure provides a method of identifying a subject having acute myeloid leukemia (AML) that will be refractory to treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) identifying that the subject will be refractory to treatment with a combination of venetoclax and azacitidine when: the sample is classified as FAB-M0, FAB-M1 or FAB-M2, the clinical karyotype is complex, the side scatter intensity is less than the side scatter intensity predetermined cutoff value, the expression level of CD11b or CD64 is greater than or equal to its corresponding predetermined cutoff value, the expression level of CD45 is less than its corresponding first predetermined cutoff value and greater than or equal to its corresponding second predetermined cutoff value, and the expression level of CD117 is greater than or equal to its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M0, FAB-M1 or FAB-M2, has a complex clinical karyotype and is CD45^{Med}/SSC^{low}/CD117+/CD11b+ or CD45^{Med}/SSC^{low}/CD117+/CD64+, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to at least one corresponding predetermined cutoff value for each biomarker; g) administering a first therapy to the subject when: the sample is classified as FAB-M0, FAB-M1 or FAB-M2, the clinical karyotype is complex, the side scatter intensity is less than the side scatter intensity predetermined cutoff value, the expression level of CD11b or CD64 is greater than or equal to its corresponding predetermined cutoff value, the expression level of CD45 is less than its corresponding first predetermined cutoff value and greater than or equal to its corresponding second predetermined cutoff value, and the expression level of CD117 is greater than or equal to its corresponding predetermined cutoff value, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having AML that will relapse after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be relapse after treatment with a combination of venetoclax and azacitidine when the sample from the subject is classified as FAB-M4, has a complex clinical karyotype and is CD45^{Bright}/SSC^{medium}/CD117−/CD11b-PartiallyPositive(PP)/CD64-PP.

The present disclosure provides a method of identifying a subject having AML that will relapse after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be relapse after treatment with a combination of venetoclax and azacitidine when: the sample is classified as FAB-M4, the clinical karyotype is complex, the side scatter intensity is greater than or equal to the first side scatter intensity predetermined cutoff value and less than the second side scatter intensity predetermined cutoff value, the expression level of each of CD45, CD11b and CD64 is greater than or equal to its corresponding predetermined cutoff value, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M4, has a complex clinical karyotype and is CD45^{Bright}/SSC^{medium}/CD117−/CD11b-PartiallyPositive(PP)/CD64-PP, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M4, the clinical karyotype is complex, the side scatter intensity is greater than or equal to the first side scatter intensity predetermined cutoff value and less than the second side scatter intensity predetermined cutoff value, the expression level of each of CD45, CD11b and CD64 is greater than or equal to its corresponding predetermined cutoff value, and the expression level of CD117 is less than its corresponding predetermined cutoff value, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having AML that will relapse after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be relapse after treatment with a combination of venetoclax and azacitidine when: the sample is classified as FAB-M4, the clinical karyotype is complex, the side scatter intensity is greater than or equal to the first side scatter intensity predetermined cutoff value and less than the second side scatter intensity predetermined cutoff value, the expression level of CD45 is greater than or equal to its corresponding predetermined cutoff value, the expression level of each of CD11b and CD64 is greater than or equal to its corresponding predetermined cutoff value such that the sample is partially positive for CD11b and CD64, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M4, the clinical karyotype is complex, the side scatter intensity is greater than or equal to the first side scatter intensity predetermined cutoff value and less than the second side scatter intensity predetermined cutoff value, the expression level of CD45 is greater than or equal to its corresponding predetermined cutoff value, the expression level of each of CD11b and CD64 is greater than or equal to its corresponding predetermined cutoff value such that the sample is partially positive for CD11b and CD64, and the expression level of CD117 is less than its corresponding predetermined cutoff value, wherein the first therapy comprises administering to the subject a therapeutically effective amount of an MCL-1 inhibitor, a therapeutically effective amount of at least one MCL-1 inhibitor in combination with a therapeutically effective amount of azacitidine, a therapeutically effective amount of an anti-AML therapy that does not comprise a combination of venetoclax and azacitidine, a therapeutically effective amount of an anti-cancer therapy, a therapeutically effective amount of chemotherapy, a therapeutically effective amount of targeted drug therapy, a therapeutically effective amount of radiation therapy, a therapeutically effective amount of immunotherapy, a stem cell transplant or any combination thereof.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be exhibit durable remission after treatment with a combination of venetoclax and azacitidine when the sample from the subject is classified as FAB-M5, has a normal clinical karyotype and is CD45^{Bright}/SSC^{high}/CD117−/CD11b+/CD64+.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be exhibit durable remission after treatment with a combination of venetoclax and azacitidine when: the sample is classified as FAB-M5, the clinical karyotype is normal, the side scatter intensity is greater than or equal to the side scatter intensity predetermined cutoff value, the expression level of CD45 is greater than or equal to its corresponding predetermined cutoff value, the expression level of each of CD45, CD11b and CD64 is greater than or equal to its corresponding first predetermined cutoff value, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M5, has a normal clinical karyotype and is CD45^{Bright}/SSC^{high}/CD117−/CD11b+/CD64+, wherein the first therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M5, the clinical karyotype is normal, the side scatter intensity is greater than or equal to the side scatter intensity predetermined cutoff value, the expression level of CD45 is greater than or equal to its corresponding predetermined cutoff value, the expression level of each of CD45, CD11b and CD64 is greater than or equal to its corresponding first predetermined cutoff value, and the expression level of CD117 is less than its corresponding predetermined cutoff value, wherein the first therapy comprises administering to the subject a therapeutically effective amount of venetoclax in combination with a therapeutically effective amount of azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be exhibit durable remission after treatment with a combination of venetoclax and azacitidine when the sample from the subject is classified as FAB-M4, has a normal clinical karyotype and is CD45^{Bright}/SSC^{medium}/CD117−/CD11b-PartiallyPositive(PP)/CD64-PP.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be exhibit durable remission after treatment with a combination of venetoclax and azacitidine when: the sample is classified as FAB-M4, the clinical karyotype is normal, the side scatter intensity is less than the first side scatter intensity predetermined cutoff value and greater than or equal to the second side scatter intensity predetermined cutoff value, the expression level of each of CD45, CD11b and CD64 is greater than or equal to its corresponding first predetermined cutoff value, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M4, has a normal clinical karyotype and is CD45^{Bright}/SSC^{medium}/CD117−/CD11b-PartiallyPositive(PP)/CD64-PP, wherein the first therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M4, the clinical karyotype is normal, the side scatter intensity is less than the first side scatter intensity predetermined cutoff value and greater than or equal to the second side scatter intensity predetermined cutoff value, the expression level of each of CD45, CD11b and CD64 is greater than or equal to its corresponding first predetermined cutoff value, and the expression level of CD117 is less than its corresponding predetermined cutoff value, wherein the first therapy comprises administering to the subject a therapeutically effective amount of venetoclax in combination with a therapeutically effective amount of azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be exhibit durable remission after treatment with a combination of venetoclax and azacitidine when: the sample is classified as FAB-M4, the clinical karyotype is normal, the side scatter intensity is less than the first side scatter intensity predetermined cutoff value and greater than or equal to the second side scatter intensity predetermined cutoff value, the expression level of CD45 is greater than or equal to its corresponding predetermined cutoff value, the expression level of each of CD11b and CD64 is greater than or equal to its corresponding predetermined cutoff value such that the sample is partially positive for CD11b and CD64, and the expression level of CD117 is less than its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a first and a second predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M4, the clinical karyotype is normal, the side scatter intensity is less than the first side scatter intensity predetermined cutoff value and greater than or equal to the second side scatter intensity predetermined cutoff value, the expression level of CD45 is greater than or equal to its corresponding predetermined cutoff value, the expression level of each of CD11b and CD64 is greater than or equal to its corresponding predetermined cutoff value such that the sample is partially positive for CD11b and CD64, and the expression level of CD117 is less than its corresponding predetermined cutoff value, wherein the first therapy comprises administering to the subject a therapeutically effective amount of venetoclax in combination with a therapeutically effective amount of azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be exhibit durable remission after treatment with a combination of venetoclax and azacitidine when the sample from the subject is classified as FAB-M0, FAB-M1 or FAB-M2, has a normal or complex clinical karyotype and is CD45^{Med}/SSC^{low}/CD117+/CD11b−/CD64−.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be exhibit durable remission after treatment with a combination of venetoclax and azacitidine when: the sample is classified as FAB-M0, FAB-M1 or FAB-M2, the clinical karyotype is normal or complex, the side scatter intensity is less than the side scatter intensity predetermined cutoff value, the expression level of CD45 is less than its corresponding first predetermined cutoff value and greater than or equal to its corresponding second predetermined cutoff value, the expression level of each of CD11b and CD64 is less than its corresponding predetermined cutoff value, and the expression level of CD117 is greater than or equal to its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M0, FAB-M1 or FAB-M2, has a normal or complex clinical karyotype and is CD45^{Med}/SSC^{low}/CD117+/CD11b−/CD64−, wherein the first therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M0, FAB-M1 or FAB-M2, the clinical karyotype is normal or complex, the side scatter intensity is less than the side scatter intensity predetermined cutoff value, the expression level of CD45 is less than its corresponding first predetermined cutoff value and greater than or equal to its corresponding second predetermined cutoff value, the expression level of each of CD11b and CD64 is less than its corresponding predetermined cutoff value, and the expression level of CD117 is greater than or equal to its corresponding predetermined cutoff value, wherein the first therapy comprises administering to the subject a therapeutically effective amount of venetoclax in combination with a therapeutically effective amount of azacitidine.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) identifying that the subject will be exhibit durable remission after treatment with a combination of venetoclax and azacitidine when the sample from the subject is classified as FAB-M0, FAB-M1 or FAB-M2, has a normal clinical karyotype and is CD45^{Med}/SSC^{low}/CD117+/CD11b+ or CD45^{Med}/SSC^{low}/CD117+/CD64+.

The present disclosure provides a method of identifying a subject having AML that will exhibit durable remission after treatment with a combination of venetoclax and azacitidine, the method comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) identifying that the subject will be exhibit durable remission after treatment with a combination of venetoclax and azacitidine when: the sample is classified as FAB-M0, FAB-M1 or FAB-M2, the clinical karyotype is normal, the side scatter intensity is less than the side scatter intensity predetermined cutoff value, the expression level of CD45 is less than its corresponding first predetermined cutoff value and greater than or equal to its corresponding second predetermined cutoff value, the expression level of CD11b or CD64 is greater than its corresponding first predetermined cutoff value, and the expression level of CD117 is greater than or equal to its corresponding predetermined cutoff value.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity (SSC) in a sample from the subject; d) determining the expression level of each of the biomarkers selected from CD45, CD117, CD11b and CD64 in a sample from the subject; e) administering a first therapy to the subject when the sample from the subject is classified as FAB-M0, FAB-M1 or FAB-M2, has a normal clinical karyotype and is CD45^{Med}/SSC^{low}/CD117+/CD11b+ or CD45^{Med}/SSC^{low}/CD117+/CD64+, wherein the first therapy comprises administering to the subject a therapeutically effective amount of venetoclax in combination with a therapeutically effective amount of azacitidine.

The present disclosure provides a method of treating acute myeloid leukemia in a subject comprising: a) classifying a sample from the subject according to the French-American-British (FAB) classification system of acute myeloid leukemia; b) determining the clinical karyotype of a sample from the subject; c) determining side scatter intensity in a sample from the subject; d) comparing the side scatter intensity to a predetermined cutoff value; e) determining the expression level of each of the biomarkers selected from CD45, CD117, CD11b and CD64 in a sample from the subject; f) comparing the expression level of each of the biomarkers to a corresponding predetermined cutoff value for each biomarker; g) administering a first therapy to the subject when: the sample is classified as FAB-M0, FAB-M1 or FAB-M2, the clinical karyotype is normal, the side scatter intensity is less than the side scatter intensity predetermined cutoff value, the expression level of CD45 is less than its corresponding first predetermined cutoff value and greater than or equal to its corresponding second predetermined cutoff value, the expression level of CD11b or CD64 is greater than its corresponding first predetermined cutoff value, and the expression level of CD117 is greater than or equal to its corresponding predetermined cutoff value, wherein the first therapy comprises administering to the subject a therapeutically effective amount of venetoclax in combination with a therapeutically effective amount of azacitidine.

The present disclosure provides a method of treating acute myeloid leukemia in a subject comprising administering to the subject a therapeutically effective amount of at least one MCL-1 inhibitor. The present disclosure provides a method of treating acute myeloid leukemia in a subject comprising administering to the subject a therapeutically effective amount of at least one MCL-1 inhibitor in combination with at least one other therapy. The present disclosure provides a method of treating acute myeloid leukemia in a subject comprising administering to the subject a therapeutically effective amount of at least one MCL-1 inhibitor in combination with a therapeutically effective amount of venetoclax and a therapeutically effective mount of azacitidine. The present disclosure provides a method of treating acute myeloid leukemia in a subject comprising administering to the subject a therapeutically effective amount of at least one MCL-1 inhibitor in combination with a therapeutically effective amount of azacitidine.

The present disclosure provides at least one MCL-1 inhibitor for use in the treatment of acute myeloid leukemia in a subject, wherein the at least one MCL-1 inhibitor is for administration to the subject in at least one therapeutically effective amount.

The present disclosure provides at least one MCL-1 inhibitor for use in the manufacture of a medicament for the treatment of acute myeloid leukemia in a subject, wherein the at least one MCL-1 inhibitor is for administration to the subject in at least one therapeutically effective amount.

The present disclosure provides a combination of at least one MCL-1 inhibitor and azacitidine for use in the treatment of acute myeloid leukemia in a subject, wherein the at least one MCL-1 inhibitor and azacitidine are for administration to the subject in at least one therapeutically effective amount each.

The present disclosure provides a combination of at least one MCL-1 inhibitor and azacitidine for use in the manufacture of a medicament for the treatment of acute myeloid leukemia in a subject, wherein the at least one MCL-1 inhibitor and azacitidine are for administration to the subject in at least one therapeutically effective amount each.

The present disclosure provides at least one MCL-1 inhibitor for use in the treatment of acute myeloid leukemia in a subject, wherein the at least one MCL-1 inhibitor is for administration to the subject in at least one therapeutically effective amount and wherein the treatment further comprises administration of at least one therapeutically effective amount of azacitidine to the subject.

The present disclosure provides at least one MCL-1 inhibitor for use in the manufacture of a medicament for the treatment of acute myeloid leukemia in a subject, wherein the at least one MCL-1 inhibitor is for administration to the subject in at least one therapeutically effective amount, and wherein the treatment further comprises administration of at least one therapeutically effective amount of azacitidine to the subject.

The present disclosure provides azacitidine for use in the treatment of acute myeloid leukemia in a subject, wherein the azacitidine is for administration to the subject in at least one therapeutically effective amount and wherein the treatment further comprises administration of at least one therapeutically effective amount of at least one MCL-1 inhibitor to the subject.

The present disclosure provides azacitidine for use in the manufacture of a medicament for the treatment of acute myeloid leukemia in a subject, wherein the azacitidine is for administration to the subject in at least one therapeutically effective amount, and wherein the treatment further comprises administration of at least one therapeutically effective amount of at least one MCL-1 inhibitor to the subject.

In some aspects of the methods of the present disclosure, identifying that a subject having AML will relapse after treatment with a combination of venetoclax and azacitidine further comprises identifying that the subject will also initially exhibit complete remission, complete remission with incomplete blood count recovery, morphologic leukemia-free state or any combination thereof after treatment with a combination of venetoclax and azacitidine.

The present disclosure provides a method of treating acute myeloid leukemia in a subject comprising: a) determining the expression level of CD7 in a sample from the subject; b) comparing the expression level of CD7 to a predetermined cutoff value; and c) administering to the subject a first therapy when the expression level of CD7 is equal to or greater than the predetermined cutoff or administering to the subject a second therapy when the expression level of CD7 is less than the predetermined cutoff, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure provides a method of treating acute myeloid leukemia in a subject comprising: a) determining the expression level of CD7 in a sample from the subject; b) comparing the expression level of CD7 to a predetermined cutoff value; and c) administering to the subject a first therapy when the expression level of CD7 is equal to or greater than the predetermined cutoff or administering to the subject a second therapy when the expression level of CD7 is less than the predetermined cutoff.

The present disclosure provides a method of treating acute myeloid leukemia in a subject comprising: a) determining if AML cells in a sample from the subject are CD7+ or CD7−; and b) administering to the subject a first therapy when the AML cells in the sample are CD7+ or administering to the subject a second therapy when the AML cells in the sample are CD7−, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure provides a method of treating acute myeloid leukemia in a subject comprising: a) determining if AML cells in a sample from the subject are CD7+ or CD7−; and b) administering to the subject a first therapy when the AML cells in the sample are CD7+ or administering to the subject a second therapy when the AML cells in the sample are CD7−.

The term “CD7+”, as used herein, is used in its broadest sense to refer to a cell that expresses the cell surface marker CD7. In some aspects, a cell is considered CD7+ if the cell expresses the cell surface marker CD7 at a level greater than a predetermined cutoff level.

The term “CD7−”, as used herein, is used in its broadest sense to refer to a cell that does not express the cell surface marker CD7. In some aspects, a cell is considered CD7− if the cell expresses the cell surface marker CD7 at a level less than a predetermined cutoff level.

The present disclosure also provides a method of providing an acute myeloid leukemia treatment recommendation comprising: a) determining the expression level of CD7 in a sample from the subject; b) comparing the expression level of CD7 to a predetermined cutoff value; and c) recommending the administration of a first therapy to the subject when the expression level of CD7 is equal to or greater than the predetermined cutoff or recommending the administration of a second therapy when the expression level of CD7 is less than the predetermined cutoff, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure also provides a method of providing an acute myeloid leukemia treatment recommendation comprising: a) determining the expression level of CD7 in a sample from the subject; b) comparing the expression level of CD7 to a predetermined cutoff value; and c) recommending the administration of a first therapy to the subject when the expression level of CD7 is equal to or greater than the predetermined cutoff or recommending the administration of a second therapy when the expression level of CD7 is less than the predetermined cutoff.

The present disclosure also provides a method of providing an acute myeloid leukemia treatment recommendation comprising a) determining if AML cells in a sample from the subject are CD7+ or CD7−; and b) recommending the administration of a first therapy to the subject when the AML, cells in the sample are CD7+ or recommending the administration of a second therapy when the AML cells in the sample are CD7−, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure also provides a method of providing an acute myeloid leukemia treatment recommendation comprising a) determining if AML cells in a sample from the subject are CD7+ or CD7−; and b) recommending the administration of a first therapy to the subject when the AML cells in the sample are CD7+ or recommending the administration of a second therapy when the AML cells in the sample are CD7−.

The present disclosure also provides a method of providing an acute myeloid leukemia treatment recommendation comprising: a) determining the expression level of CD7 in a sample from the subject; b) comparing the expression level of CD7 to a predetermined cutoff value; and c) producing a report, wherein the report recommends the administration of a first therapy to the subject when the expression level of CD7 is equal to or greater than the predetermined cutoff or the report recommends the administration of a second therapy when the expression level of CD7 is less than the predetermined cutoff, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure also provides a method of providing an acute myeloid leukemia treatment recommendation comprising: a) determining the expression level of CD7 in a sample from the subject; b) comparing the expression level of CD7 to a predetermined cutoff value; and c) producing a report, wherein the report recommends the administration of a first therapy to the subject when the expression level of CD7 is equal to or greater than the predetermined cutoff or the report recommends the administration of a second therapy when the expression level of CD7 is less than the predetermined cutoff.

The present disclosure also provides a method of providing an acute myeloid leukemia treatment recommendation comprising a) determining if AML, cells in a sample from the subject are CD7+ or CD7−; and b) producing a report, wherein the report recommends the administration of a first therapy to the subject when the AML cells in the sample are CD7+ or recommends the administration of a second therapy when the AML cells in the sample are CD7−, wherein the first therapy comprises administering to the subject a therapeutically effective amount of an immunotherapy, and wherein the second therapy comprises administering to the subject a therapeutically effective amount of venetoclax in combination with a therapeutically effective amount of azacitidine.

The present disclosure also provides a method of providing an acute myeloid leukemia treatment recommendation comprising a) determining if AML cells in a sample from the subject are CD7+ or CD7−; and b) producing a report, wherein the report recommends the administration of a first therapy to the subject when the AML cells in the sample are CD7+ or recommends the administration of a second therapy when the AML cells in the sample are CD7−.

The present disclosure also provides a method of the prognosis of acute myeloid leukemia in a subject comprising: a) determining the expression level of CD7 in a sample from the subject; b) comparing the expression level of CD7 to a predetermined cutoff value; and c) identifying the subject as having a poor prognosis when the expression level of CD7 is equal to or greater than the predetermined cutoff or identifying the subject as having a good prognosis when the expression level of CD7 is less than the predetermined cutoff.

The present disclosure also provides a method of the prognosis of acute myeloid leukemia in a subject comprising a) determining if AML cells in a sample from the subject are CD7+ or CD7−; and b) identifying the subject as having a poor prognosis when the AML cells in the sample are CD7+ or identifying the subject as having a good prognosis the AML cells in the sample are CD7−.

The present disclosure also provides a method of the prognosis of acute myeloid leukemia in a subject comprising: a) determining the expression level of CD7 in a sample from the subject; b) comparing the expression level of CD7 to a predetermined cutoff value; and c) producing a report, wherein the report identifies the subject as having a poor prognosis when the expression level of CD7 is equal to or greater than the predetermined cutoff or the report identifies the subject as having a good prognosis when the expression level of CD7 is less than the predetermined cutoff.

The present disclosure also provides a method of the prognosis of acute myeloid leukemia in a subject comprising a) determining if AML cells in a sample from the subject are CD7+ or CD7−; and b) producing a report, wherein the report identifies the subject as having a poor prognosis when the AML cells in the sample are CD7+ or identifies the subject as having a good prognosis the AML cells in the sample are CD7−.

The present disclosure also provides a method of identifying the risk of relapse of acute myeloid leukemia in a subject comprising: a) determining the expression level of CD7 in a sample from the subject; b) comparing the expression level of CD7 to a predetermined cutoff value; and c) identifying the subject as having a high risk of relapse when the expression level of CD7 is equal to or greater than the predetermined cutoff or identifying the subject as having a low risk of relapse when the expression level of CD7 is less than the predetermined cutoff.

The present disclosure also provides a method of providing an acute myeloid leukemia treatment recommendation comprising a) determining if AML cells in a sample from the subject are CD7+ or CD7−; and b) identifying the subject as having a high risk of relapse when the AML cells in the sample are CD7+ or identifying the subject as having a low risk of relapse when the AML cells in the sample are CD7−.

The present disclosure also provides a method of identifying the risk of relapse of acute myeloid leukemia in a subject comprising: a) determining the expression level of CD7 in a sample from the subject; b) comparing the expression level of CD7 to a predetermined cutoff value; and c) producing a report, wherein the report identifies the subject as having a high risk of relapse when the expression level of CD7 is equal to or greater than the predetermined cutoff or the report identifies the subject as having a low risk of relapse when the expression level of CD7 is less than the predetermined cutoff.

The present disclosure also provides a method of providing an acute myeloid leukemia treatment recommendation comprising a) determining if AML cells in a sample from the subject are CD7+ or CD7−; and b) producing a report, wherein the report identifies the subject as having a high risk of relapse when the AML cells in the sample are CD7+ or identifies the subject as having a low risk of relapse when the AML cells in the sample are CD7−.

In some aspects, determining that AML cells in a sample are CD7+ can comprise determining that at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% or at least about 99% of the AML cells in the sample express CD7.

In some aspects, determining that AML cells in a sample are CD7+ can comprise determining that at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% or at least about 99% of the AML cells in the sample express CD7 at a level greater than a predetermined cutoff level.

In some aspects, determining that AML cells in a sample are CD7− can comprise determining that at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% or at least about 99% of the AML cells in the sample do not express CD7.

In some aspects, determining that AML cells in a sample are CD7− can comprise determining that at least about 1%, or at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95% or at least about 99% of the AML, cells in the sample express CD7 at a level less than a predetermined cutoff level.

The present disclosure provides a method of identifying a subject having acute myeloid leukemia (AML) that will be refractory to treatment with a combination of venetoclax and azacitidine, the method comprising: a) determining the expression level of CD7 in a sample from the subject; b) comparing the expression level of CD7 to a predetermined cutoff value; and c) identifying that the subject will be refractory to treatment with a combination of venetoclax and azacitidine when the expression level of CD7 is equal to or greater than the predetermined cutoff.

The present disclosure provides a method of identifying a subject having acute myeloid leukemia (AML) that will relapse from treatment with a combination of venetoclax and azacitidine, the method comprising: a) determining the expression level of CD7 in a sample from the subject; b) comparing the expression level of CD7 to a predetermined cutoff value; and c) identifying that the subject will be refractory to treatment with a combination of venetoclax and azacitidine when the expression level of CD7 is equal to or greater than the predetermined cutoff.

The present disclosure provides a method of treating CD7+ acute myeloid leukemia in a subject comprising administering to the subject a therapeutically effective amount of an immunotherapy. In some aspects, an immunotherapy can comprise a therapeutically effective amount of at least one antibody, at least one checkpoint inhibitor, at least one CAR-T cell, or any combination thereof. In some aspects, an at least one CAR-T cell can comprise a chimeric antigen receptor, wherein the chimeric antigen receptor comprises an antigen binding domain, wherein the antigen binding domain binds to CD7.

In some aspects of the methods of the present disclosure, a predetermined cutoff value can be determined by analyzing one or more control samples. In some aspects, the one or more control samples are samples from subjects having AML who have already been treated with a combination of venetoclax and azacitidine, such that it is known whether the AML in those subjects is refractory to treatment with a combination of venetoclax and azacitidine, responds to treatment with a combination of venetoclax and azacitidine, exhibits durable remission after treatment with a combination of venetoclax and azacitidine or relapses after treatment with a combination of venetoclax and azacitidine.

In some aspects, the one or more control samples can comprise samples from subjects who are diagnosed with AML, from subjects who are healthy (e.g. do not have AML), from subjects having AML that is refractory to treatment with a combination of venetoclax and azacitidine, from subjects having AML that responds to treatment with a combination of venetoclax and azacitidine, from subjects having AML that have relapsed after treatment with a combination venetoclax and azacitidine, from subject having AML that have exhibited durable remission after treatment with a combination of venetoclax and azacitidine, from subjects having AML that have a poor prognosis or any combination thereof.

The present disclosure provides a method of treating CD7+ acute myeloid leukemia in a subject comprising administering to the subject at least one therapeutically effective amount of a composition comprising a plurality of CAR-T cells, wherein the at least one CAR-T cell in the plurality comprises a chimeric antigen receptor, wherein the chimeric antigen receptor comprises an antigen binding domain, wherein the antigen binding domains binds to CD7.

The present disclosure provides a composition comprising a plurality of CAR-T cells, wherein the at least one CAR-T cell in the plurality comprises a chimeric antigen receptor, wherein the chimeric antigen receptor comprises an antigen binding domain, wherein the antigen binding domains binds to CD7, for use in the treatment of CD7+ acute myeloid leukemia in a subject, wherein the composition is for administration to the subject in at least on therapeutically effective amount.

The present disclosure provides a composition comprising a plurality of CAR-T cells, wherein the at least one CAR-T cell in the plurality comprises a chimeric antigen receptor, wherein the chimeric antigen receptor comprises an antigen binding domain, wherein the antigen binding domains binds to CD7, for use in the manufacture of a medicament for the treatment of CD7+ acute myeloid leukemia in a subject, wherein the composition is for administration to the subject in at least on therapeutically effective amount.

In some aspects the methods of the present disclosure can further comprise identifying that the subject having AML that will relapse after treatment with a combination of venetoclax and azacitidine will also initially exhibit complete remission, complete remission with incomplete blood count recovery, morphologic leukemia-free state or any combination thereof after treatment with a combination of venetoclax and azacitidine.

In some aspects, the methods of the present disclosure can further comprise administering at least one therapeutically effective amount of at least one alternative therapy to the subject that is identified as a subject that will be refractory to or that will relapse from treatment with a combination of venetoclax and azacitidine, wherein the at least one alternative therapy does not comprise venetoclax in combination with azacitidine.

In some aspects, the methods of the present disclosure can further comprise providing a treatment recommendation to the subject that is identified as a subject that will be refractory to or that will relapse from treatment with a combination of venetoclax and azacitidine, wherein the treatment recommendation comprises recommending the administration of at least one therapeutically effective amount of at least one alternative therapy.

In some aspects, an least one alternative therapy can comprise anti-cancer therapy, chemotherapy, targeted drug therapy, radiation therapy, immunotherapy, stem cell transplant or any combination thereof.

In some aspects, the methods of the present disclosure can further comprise administering at least one therapeutically effective amount of venetoclax in combination with a therapeutically effective amount of azacitidine to the subject that is identified as a subject that will exhibit durable remission after treatment with a combination of venetoclax and azacitidine.

In some aspects, the methods of the present disclosure can further comprise providing a treatment recommendation to the subject that is identified as a subject that will exhibit durable remission after treatment with a combination of venetoclax and azacitidine, wherein the treatment recommendation comprises recommending the administration of at least one therapeutically effective amount of venetoclax in combination with a therapeutically effective amount of azacitidine.

In some aspects of the methods of the present disclosure, a sample can comprise acute myeloid leukemia cells. Acute myeloid leukemia cells can comprise acute myeloid leukemia blast cells, leukemia stem cells or a combination thereof. Leukemia stem cells can comprise reactive oxygen species-low leukemia stem cells.

In some aspects of the methods of the present disclosure, a sample can be blood, bone marrow biopsy, a bone marrow aspirate, a biopsy of a chloroma, a tissue biopsy, cerebrospinal fluid or any combination thereof. Samples can be isolated from a subject using methods known in the art. In a non-limiting example, a cerebrospinal fluid sample can be isolated from a subject by performing a lumbar puncture (spinal tap). In another non-limiting example, a bone marrow biopsy or a bone marrow aspirate can be isolated by using a needle to pierce a bone, such as a hip bone, to obtain bone marrow.

In some aspects of the methods of the present disclosure, a subject can have been previously diagnosed with acute myeloid leukemia. In some aspects, a subject can have been previously administered an initial therapy. The subject may have not responded to the initial therapy or may have only partially responded to the initial therapy. In some aspects, a subject can have relapsed acute myeloid leukemia.

In some aspects, a response to a therapy in a subject can be evaluated using methods known in the art. In a non-limiting example, a response to a therapy can be evaluated by isolating a sample from the subject (be plasma, serum, blood, bone marrow biopsy, a bone marrow aspirate, a biopsy of a chloroma, a tissue biopsy, cerebrospinal fluid or any combination thereof) and analyzing the sample to determine the concentration of leukemia cells, markers or combination thereof.

In some aspects of the methods of the present disclosure, an initial therapy can comprise administering to the subject a therapeutically effective amount of venetoclax in combination with a therapeutically effective amount of azacitidine. In some aspects of the methods of the present disclosure, an initial therapy can comprise administering to a subject a therapeutically effective amount of an anti-cancer therapy, chemotherapy, targeted drug therapy, radiation therapy, immunotherapy, stem cell transplant or any combination thereof.

In some aspects of the methods of the present disclosure, a first therapy can comprise administering to a subject a therapeutically effective amount of an anti-cancer therapy, chemotherapy, targeted drug therapy, radiation therapy, immunotherapy, stem cell transplant or any combination thereof.

In some aspects of the methods of the present disclosure, a second therapy can comprise administering to a subject a therapeutically effective amount of an anti-cancer therapy, chemotherapy, targeted drug therapy, radiation therapy, immunotherapy, stem cell transplant or any combination thereof.

In some aspects of the methods of the present disclosure, targeted drug therapy can comprise the administration of compounds that specifically target the cellular malfunctions that allow cancer cells to grow and proliferate. In some aspects of the methods of the present disclosure, targeted drug therapy can comprise administering to a subject a therapeutically effective amount of at least one agent that modulates a cellular pathway, wherein the cellular pathway is a pathway set forth in Table 1. In some aspects of the methods of the present disclosure, a targeted drug therapy can comprise administering to a subject a therapeutically effective amount of venetoclax in combination with a therapeutically effective amount of azacitidine.

In some aspects of the methods of the present disclosure, targeted drug therapy can comprise administering to a subject a therapeutically effective amount of an MCL-1 inhibitor. MCL-1 inhibitors can include, but are not limited to, YM155, VU103 or any combination thereof. Targeted drug therapy can comprising administering to a subject a therapeutically effective amount of an MCL-1 inhibitor in combination with a therapeutically effective amount of azacitidine. Targeted drug therapy can comprise administering to a subject a therapeutically effective amount of an MCL-1 inhibitor in combination with at least one hypomethylating agents. Hypomethylating agents can include, but are not limited to azacitidine, cytarabine, decitabine and any other hypomethylating agent known in the art. A metabolism modulating agent can be a BCL-2 inhibitor. BCL-2 inhibitors can include, but are not limited to, venetoclax, navitoclax, and any other BCL-2 inhibitor known in the art. In some aspects of any of the methods of the present disclosure, azacitidine can be substituted with at least one other hypomethylating agent, including, but not limited to azacitidine, cytarabine, decitabine and any other hypomethylating agent known in the art.

TABLE 1
Cellular Pathways

| Pathways | Pathways |
|---|---|
| Lysosome pathways | amino acid uptake pathways |
| nuclear import pathways | fatty acid oxidation pathways |
| PI3 Kinase and Akt signaling pathways | myc signaling pathways |
| NF-kB signaling pathways | Proteasome pathways |
| Autophagy pathways | nicotinamide metabolism (NAMPT enzyme, |
| | other regulators) pathways |
| Mitophagy pathways | amino acid catabolism pathways |
| FIS1 signaling pathways | AMPK signaling pathways |
| CD38 pathways | protein synthesis pathways |
| nicotinamide metabolism pathways | Nampt pathways |
| Stress response pathways | Energy metabolism |
| DNA repair pathway | CD36 fatty acid oxidation pathway |
| DNA methylation | BCL-2 activity |

In some aspects of the methods of the present disclosure, a subject can be at least about 5 years of age, or at least about 10 years of age, or at least about 15 years of age, or at least about 18 years of age, or at least about 20 years of age, or at least about 25 years of age, or at least about 30 years of age, or at least about 35 years of age, or at least about 40 years of age, or at least about 45 years of age, or at least about 50 years of age, or at least about 55 years of age, or at least about 60 years of age, or at least about 65 years of age, or at least about 70 years of age, or at least about 75 years of age, or at least about 80 years of age, or at least about 85 years of age, or at least about 90 years of age, or at least about 95 years of age, or at least about 100 years of age.

In some aspects of the methods of the present disclosure, the expression of biomarkers (e.g. CD45, CD117, CD11b, CD64 and CD7) can be determined using methods known in the art. These methods include, but are not limited to, PCR, high-throughput sequencing, next generation sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

In some aspects of the methods of the present disclosure, classifying a sample from the subject according to the French-American-British (FAB) classification system of acute myeloid leukemia can comprise histological techniques standard in the art. The traditional French-American-British (FAB) classification of AML is as follows: M0—Undifferentiated acute myeloblastic leukemia; M1—Acute myeloblastic leukemia with minimal maturation; M2—Acute myeloblastic leukemia with maturation; M3—Acute promyelocytic leukemia (APL); M4—Acute myelomonocytic leukemia; M4 eos—Acute myelomonocytic leukemia with eosinophilia; M5—Acute monocytic leukemia; M6—Acute erythroid leukemia; and M7—Acute megakaryoblastic leukemia.

In some aspects of the methods of the present disclosure, determining the clinical karyotype of a sample from the subject can comprise cytogenetic methods standard in the art.

In some aspects of the methods of the present disclosure, the methods can further comprise determining the expression level of the biomarker CD68.

In some aspects of the methods of the present disclosure, determining the side scatter intensity in a sample from the subject can comprise flow cytometry techniques standard in the art.

In some aspects of the methods of the present disclosure, a complex clinical karyotype can be defined as the presence of at least 3 chromosomal aberrations. In some aspects of the methods of the present disclosure, a complex clinical karyotype can be defined as at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90 or at least about 100 chromosomal aberrations.

In some aspects of the methods of the present disclosure, a normal clinical karyotype can be defined as 46 XY karyotype. In some aspects of the methods of the present disclosure can be defined as a 46 XX karyotype. In some aspects of the methods of the present disclosure, a normal clinical karyotype can be defined as a 46 XY karyotype or a 46 XX karyotype.

In a non-limiting example, the expression of CD45 can be determined using an antibody or antibody fragment that binds to CD45. CD45 can comprise any portion of the amino acid sequence set forth in SEQ ID NO: 1. The antibody or antibody fragment can be labeled, directly or indirectly. For example, the antibody or antibody fragment can be labeled directly or indirectly with a fluorescent moiety.

In another non-limiting example, the expression of CD45 can be determined using at least one nucleic acid probe comprising a sequence complementary to CD45 mRNA. CD45 mRNA can comprise any portion of the sequence set forth in SEQ ID NO: 2. The at least one nucleic acid probe can be labeled, directly or indirectly. For example, the at least one nucleic acid probe can be labeled directly or indirectly with a fluorescent moiety.

In a non-limiting example, the expression of CD117 can be determined using an antibody or antibody fragment that binds to CD117. CD117 can comprise any portion of the amino acid sequence set forth in SEQ ID NO: 3. The antibody or antibody fragment can be labeled, directly or indirectly. For example, the antibody or antibody fragment can be labeled directly or indirectly with a fluorescent moiety.

In another non-limiting example, the expression of CD117 can be determined using at least one nucleic acid probe comprising a sequence complementary to CD117 mRNA. CD117 mRNA can comprise any portion of the sequence set forth in SEQ ID NO: 4. The at least one nucleic acid probe can be labeled, directly or indirectly. For example, the at least one nucleic acid probe can be labeled directly or indirectly with a fluorescent moiety.

In a non-limiting example, the expression of CD11b can be determined using an antibody or antibody fragment that binds to CD11b. CD11b can comprise any portion of the amino acid sequence set forth in SEQ ID NO: 5. The antibody or antibody fragment can be labeled, directly or indirectly. For example, the antibody or antibody fragment can be labeled directly or indirectly with a fluorescent moiety.

In another non-limiting example, the expression of CD11b can be determined using at least one nucleic acid probe comprising a sequence complementary to CD11b mRNA. CD11b mRNA can comprise any portion of the sequence set forth in SEQ ID NO: 6. The at least one nucleic acid probe can be labeled, directly or indirectly. For example, the at least one nucleic acid probe can be labeled directly or indirectly with a fluorescent moiety.

In a non-limiting example, the expression of CD64 can be determined using an antibody or antibody fragment that binds to CD64. CD64 can comprise any portion of the amino acid sequence set forth in SEQ ID NO: 7. The antibody or antibody fragment can be labeled, directly or indirectly. For example, the antibody or antibody fragment can be labeled directly or indirectly with a fluorescent moiety.

In another non-limiting example, the expression of CD64 can be determined using at least one nucleic acid probe comprising a sequence complementary to CD64 mRNA. CD64 mRNA can comprise any portion of the sequence set forth in SEQ ID NO: 8. The at least one nucleic acid probe can be labeled, directly or indirectly. For example, the at least one nucleic acid probe can be labeled directly or indirectly with a fluorescent moiety.

In a non-limiting example, the expression of CD68 can be determined using an antibody or antibody fragment that binds to CD68. CD68 can comprise any portion of the amino acid sequence set forth in SEQ ID NO: 9. The antibody or antibody fragment can be labeled, directly or indirectly. For example, the antibody or antibody fragment can be labeled directly or indirectly with a fluorescent moiety.

In another non-limiting example, the expression of CD68 can be determined using at least one nucleic acid probe comprising a sequence complementary to CD68 mRNA. CD68 mRNA can comprise any portion of the sequence set forth in SEQ ID NO: 10. The at least one nucleic acid probe can be labeled, directly or indirectly. For example, the at least one nucleic acid probe can be labeled directly or indirectly with a fluorescent moiety.

In a non-limiting example, the expression of CD7 can be determined using an antibody or antibody fragment that binds to CD7. CD7 can comprise the amino acid sequence set forth in SEQ ID NO: 11. The antibody or antibody fragment can be labeled, directly or indirectly. For example, the antibody or antibody fragment can be labeled directly or indirectly with a fluorescent moiety.

In another non-limiting example, the expression of CD7 can be determined using at least one nucleic acid probe comprising a sequence complementary to CD7 mRNA. CD7 mRNA can comprise any portion of the sequence set forth in SEQ ID NO: 12. The at least one nucleic acid probe can be labeled, directly or indirectly. For example, the at least one nucleic acid probe can be labeled directly or indirectly with a fluorescent moiety.

In some aspects of the methods of the present disclosure, immunotherapy can comprise administering a therapeutically effective amount of at least one antibody, at least one checkpoint inhibitor, at least one chimeric antigen receptor-modified T-Cell (CAR-T cell, or any combination thereof. Immunotherapy can comprise adoptive cell transfer therapy. In some aspects of the methods of the present disclosure, immunotherapy can comprise administering a therapeutically effective amount of at least one antibody, wherein the at least one antibody binds to at least one AML cell surface protein. In some aspects of the methods of the present disclosure, immunotherapy can comprise administering a therapeutically effective amount of at least one antibody, wherein the at least one antibody binds specifically to at least one AML cell surface protein.

In some aspects of the methods of the present disclosure, immunotherapy can comprise administering checkpoint inhibitors. Checkpoint inhibitors can comprise antibodies. Checkpoint inhibitors include, but are not limited to, anti-CTLA4 antibodies, anti-PD-1 antibodies, anti-PD-L1 antibodies, anti-A2AR antibodies, anti-B7-H3 antibodies, anti-B7-H4 antibodies, anti-BTLA antibodies, anti-IDO antibodies, anti-KIR antibodies, anti-LAG3 antibodies, anti-TIM3 antibodies and anti-VISTA (V-domain Ig suppressor of T cell activation) antibodies.

Anti-CTLA4 antibodies can include, but are not limited to, ipilimumab, tremelimumab and AGEN-1884. Anti-PD-1 antibodies include, but are not limited to, pembrolizumab, nivolumab pidilizumab, cemiplimab, REGN2810, AMP-224, MEDI0680, PDR001 and CT-001. Anti-PD-L1 antibodies include, but are not limited to atezolizumab, avelumab and durvalumab. Anti-CD137 antibodies include, but are not limited to, urelumab. Anti-B7-H3 antibodies include, but are not limited to, MGA271. Anti-KIR antibodies include, but are not limited to, Lirilumab. Anti-LAG3 antibodies include, but are not limited to, BMS-986016.

The term “immunotherapy” can refer to activating immunotherapy or suppressing immunotherapy. As will be appreciated by those in the art, activating immunotherapy refers to the use of a therapeutic agent that induces, enhances, or promotes an immune response, including, e.g., a T cell response while suppressing immunotherapy refers to the use of a therapeutic agent that interferes with, suppresses, or inhibits an immune response, including, e.g., a T cell response. Activating immunotherapy may comprise the use of checkpoint inhibitors. Activating immunotherapy may comprise administering to a subject a therapeutic agent that activates a stimulatory checkpoint molecule. Stimulatory checkpoint molecules include, but are not limited to, CD27, CD28, CD40, CD122, CD137, OX40, GITR and ICOS. Therapeutic agents that activate a stimulatory checkpoint molecule include, but are not limited to, MEDI0562, TGN1412, CDX-1127, lipocalin.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An antibody that binds to a target refers to an antibody that is capable of binding the target with sufficient affinity such that the antibody is useful as a diagnostic and/or therapeutic agent in targeting the target. In one embodiment, the extent of binding of an anti-target antibody to an unrelated, non-target protein is less than about 10% of the binding of the antibody to target as measured, e.g., by a radioimmunoassay (RIA) or biacore assay. In certain embodiments, an antibody that binds to a target has a dissociation constant (Kd) of <1 μM, <100 nM, <10 nM, <1 nM, <0.1 nM, <0.01 nM, or <0.001 nM (e.g. 10⁸ M or less, e.g. from 10⁸ M to 10¹³ M, e.g., from 10⁹ M to 10¹³ M). In certain embodiments, an anti-target antibody binds to an epitope of a target that is conserved among different species.

A “blocking antibody” or an “antagonist antibody” is one that partially or fully blocks, inhibits, interferes, or neutralizes a normal biological activity of the antigen it binds. For example, an antagonist antibody may block signaling through an immune cell receptor (e.g., a T cell receptor) so as to restore a functional response by T cells (e.g., proliferation, cytokine production, target cell killing) from a dysfunctional state to antigen stimulation.

An “agonist antibody” or “activating antibody” is one that mimics, promotes, stimulates, or enhances a normal biological activity of the antigen it binds. Agonist antibodies can also enhance or initiate signaling by the antigen to which it binds. In some embodiments, agonist antibodies cause or activate signaling without the presence of the natural ligand. For example, an agonist antibody may increase memory T cell proliferation, increase cytokine production by memory T cells, inhibit regulatory T cell function, and/or inhibit regulatory T cell suppression of effector T cell function, such as effector T cell proliferation and/or cytokine production.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

CAR-T cells are T cells that are genetically modified to stably express at least one chimeric antigen receptor (CAR). A CAR can comprise an extracellular domain, transmembrane domain and a cytoplasmic domain. A CAR can comprise an antigen binding domain. An antigen binding domain can be located in an extracellular domain. In some aspects of the methods of the present disclosure, the antigen binding domain binds to at least one AML cell surface protein. In some aspects of the methods of the present disclosure, the antigen binding domain binds to CD7. A CAR can also comprise an extracellular spacer (hinge) domain. An extracellular spacer can be located in an extracellular domain. A CAR can comprise a signaling domain. A signaling domain can be a T-cell activation domain. A signaling domain can be located in a cytoplasmic domain. A CAR can comprise at least one costimulatory domain. A CAR can comprise at least two costimulatory domains. A CAR can comprise at least three costimulatory domains. A costimulatory domain can be located in a cytoplasmic domain.

In some aspects of the methods of the present disclosure CAR-T cells can be autologous with respect to a subject. In some aspects, CAR-T cells can be allogeneic with respect to a subject.

In some aspects of the methods of the present disclosure, CAR-T cells may be administered either alone, or as a pharmaceutical composition in combination with diluents and/or with other components such as IL-2 or other cytokines or cell populations. Briefly, pharmaceutical compositions can comprise a plurality of CAR-T cells in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. CAR-T cells and related compositions can be administered to a subject intravenously.

A CAR-T cell can comprise a chimeric antigen receptor. A chimeric antigen receptor can comprise an antigen binding domain. An antigen binding domain can bind to CD7.

A first therapy can comprise administering to the subject a therapeutically effective amount of an immunotherapy, a stem cell transplant, anti-cancer therapy, chemotherapy, targeted drug therapy, radiation therapy, or any combination thereof.

In methods of the present disclosure, venetoclax may be administered orally. Venetoclax may be administered in a ramp-up schedule fashion over the course of 5 weeks, wherein during the first week 20 mg of venetoclax is administered daily, during the second week 50 mg of venetoclax is administered daily, during the third week 100 mg of venetoclax is administered daily, during the fourth week 200 mg of venetoclax is administered daily and during the fifth week and onwards until the end of treatment 400 mg of venetoclax is administered daily (final dose amount). Alternatively, the final dose of venetoclax can be about 300 to about 1400 mg daily. The final dose amount of venetoclax can be 400 mg daily. Alternatively, the final dose amount of venetoclax can be 800 mg daily. Alternatively still, the final dose amount of venetoclax can be 1200 mg daily. During the ramp-up schedule, the dose of venetoclax administered during any of the first, second, third or fourth weeks can be adjusted to be about 20 mg, about 50 mg, about 100 mg and about 200 mg respectively.

Azacitidine can be administered intravenously or subcutaneously. Azacitidine can be administered at a concentration of about 75 mg/m² daily for about 7 days about every 4 weeks. Alternatively, Azacitidine can be administered at a concentration of about 100 mg/m² daily for about 7 days about every 4 weeks.

In alternative aspects, Azacitidine can be administered orally. Azacitidine can be administered orally at a concentration of about 10 mg, or about 25 mg, or about 50 mg, or about 75 mg, or about 100 mg, or about 120 mg, or about 150 mg, or about 200 mg, or about 250 mg, or about 300 mg, or about 350 mg, or about 400 mg, or about 450 mg, or about 480 mg, or about 500 mg, or about 550 mg, or about 600 mg daily for about 7 days about every 4 weeks, or about 14 days about every 4 weeks, or about 21 days about every 4 weeks.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, and leukemia. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer or cervical cancer.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “refractory” as used herein, is used in its broadest sense to refer to instances in which the disease present in a subject does not respond to a particular therapy, i.e. the therapy provides no or decreased clinical benefit to that particular subject.

### EXAMPLES

### Example 1—AML Patients Who are Refractory or Relapse after Venetoclax+Azacitidine (VEN+AZA) Therapy are Phenotypically Monocytic

Samples from 75 newly diagnosed AML patients (herein referred to as Trial Cohort 1) who received venetoclax+azacitidine combination therapy (herein referred to VEN+AZA) were analyzed to determine if there are clinical features related to differentiation status that predict response to treatment with VEN+AZA, particularly features that predict a refractory response to treatment. The median age of the cohort was 72; 16/75 (21%) had a documented antecedent hematological disorder and 6/75 (8%) had treatment-related AML. 45/75 (60%) had adverse risk disease features according to the European Leukemia Net criteria. All baseline characteristics are listed in Table 1.

TABLE 1
Baseline characteristics

| | | Univariate Analysis as | Multivariate Analysis |
|---|---|---|---|
| | | a Predictor for | as a Predictor for |
| | | Refractory Disease | Refractory Disease |
| | | (p-value with 95% | (p-value with 95% |
| Baseline Variables | Value | confidence interval) | confidence interval) |
| Age (median) | 72.3 | 0.82 (0.925, 1.063) | 0.41 (0.946, 1.144) |
| Antecedent | 16 (21.0%) | 0.54 (0.057, 4.43) | 0.995 (0, —) |
| hematologic disorder | | | |
| Treatment-related | 6 (7.9%) | 0.08 (0.804, 35.391) | Not estimable |
| AML | | | |
| European Leukemia | | | |
| Network Prognostic | | | |
| Group | | | |
| Favorable | 17 (22.4%) | | |
| Intermediate | 13 (17.1%) | | |
| Adverse | 45 (59.2%) | | |
| Adverse vs. | | 0.20 (0.646, 7.947) | 0.35 (0.483, 7.957) |
| Intermediate | | | |
| TP53 | 7 (9.2%) | 0.75 (0.152, 13.875) | 0.83 (0.01, 42.067) |
| PTPN11 | 6 (7.9%) | 0.006 (2.063, 81,919) | 0.18 (0.342, 331.986) |
| IDH1/IDH2 | 22 (29.0%) | 0.99 (0, —) | |
| FLT3 ITD | 11 (14.5%) | 0.99 (0. —) | |
| ASXL1 | 16 (21.1%) | 0.001 (3.014, 97.006) | |
| French-American- | | | |
| British Classification | | | |
| M0/M1 | 57 (75.0%) | | |
| M2 | 1 (1.3%) | | |
| M4 | 7 (9.2%) | | |
| M5 | 10 (13.2%) | | |
| M6a | 1 (1.3%) | | |
| M0/M1 vs. M5 | | 0.003 (2.756, 121.973) | 0.033 (1.197, 66.644) |

Univariate analysis showed the presence of ASXL1 (p=0.001) and FAB-M5 (p=0.003) to be associated with disease that was refractory to VEN+AZA (Table 1). A multivariate analysis showed only FAB-M5 status to be significant (p=0.014, Table 1). Given that FAB-M5 is known as AML with monocytic differentiation, these findings indicate a strong correlation between myeloid differentiation status and resistance to venetoclax.

The monocytic nature of FAB-M5 patients were verified, as defined by morphology. Previous studies have shown that FAB-M5 has unique expression of the monocytic markers CD11b, CD14 and CD64, and the loss of the primitive marker CD117 and gain of CD68 have also been reported in monocytic AML. Therefore, a multicolor flow cytometry panel was designed including CD117, CD11b, CD68 and CD64 to distinguish patients with monocytic from primitive (FAB-M0/M1/M2) AML. The tumor presented as a single dominant disease population that was phenotypically primitive as evidenced by CD45-medium/SSC-low/CD117+/CD11b−/CD68−. In contrast, a typical FAB-M5 patient who was refractory to VEN+AZA (Patient-72) presented with dominant monocytic disease that was CD45-bright/SSC-high/CD117−/CD11b+/CD68+.

In a subset of AML patients, the co-existence at diagnosis of both phenotypically primitive and monocytic populations was observed, which is herein referred to as “MPM” AML (for Mixed Primitive/Monocytic). This finding is consistent with a recent study showing AML can contain a mixture of transcriptionally defined primitive and monocytic leukemic cells when analyzed by single cell RNA-seq analysis. Two MPM AML patients (Patient-12, Patient-65), both of whom had a mixture of phenotypically primitive and monocytic disease at diagnosis, were analyzed. After treatment with VEN+AZA, and subsequent relapse after response, both patients showed almost complete loss of the primitive subpopulation, and relapsed with a dominant monocytic disease that was CD45-bright/SSC-high/CD117−/CD11b+/CD68+, evidence that VEN+AZA resulted in striking selection of the monocytic population at relapse. In a third MPM AML patient, who was refractory to VEN+AZA therapy, a similar rapid selection for the monocytic sub-population was observed.

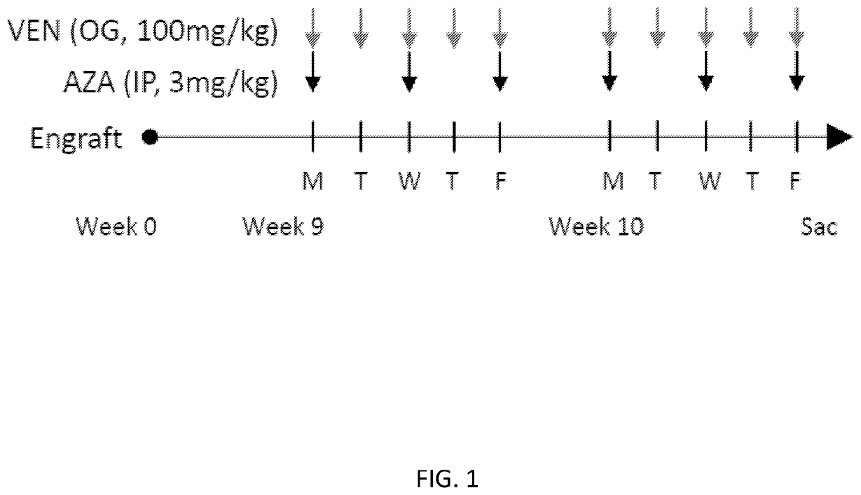

In a subsequent experiment, the selection of a monocytic phenotype was recapitulated in a patient-derived xenograft (PDX) model where the development of primary AML can be monitored with or without selective pressure from VEN+AZA. To this end, an MPM primary AML sample with approximately 1:1 ratio of primitive and monocytic disease was identified. Each population was isolated by flow cytometric sorting and subjected to karyotyping. This analysis confirmed that both populations had identical cytogenetic abnormalities and were therefore derived from the same parental population. Each population was then transplanted into cohorts of NSG-S immune deficient mice and treated with vehicle control or VEN+AZA as outlined in FIG. 1.

Post-treatment, engrafted human CD45 cells in the control group presented as two separate populations: one with a high SSC profile that was CD34−/CD11b+/CD64+(monocytic-like), and one with a low SSC profile that was CD34+/CD11b−/CD64− (primitive-like). Although VEN+AZA did not significantly reduce total human leukemic burden, the VEN+AZA treatment clearly selected out the monocytic sub-population as the ratio of monocytic to primitive gated cells was increased by more than six-fold in VEN+AZA treated mice. These findings recapitulate the myeloid selection seen in patient Patient-75 validate the xenograft model for studies of population dynamics in response to VEN+AZA treatment.

### Example 2—Monocytic Selection is Unique to VEN+AZA

The acquisition of a more mature myeloid phenotype is not a common observation in response to leukemia therapy. This observation indicates developmental plasticity at the level of AML stem cell populations. To determine if monocytic selection is unique to VEN+AZA therapy, we compared the immunophenotype of six pairs of diagnostic/relapse specimens from AML patients treated with conventional intensive induction chemotherapy. The results of this analysis are shown in Table 20

TABLE 2
Diagnostic/relapse specimen analysis

| Patient | Stage | Phenotype | CD117 | CD34 | CD11b | CD14 | CD64 |
|---|---|---|---|---|---|---|---|
| 1 | Dx | primitive + | POS | NEG | POS | NEG | POS |
| | | mono | | | | | |
| | Relapse | primitive | POS | NEG | Dim | NEG | NEG |
| 2 | Dx | mono | NEG | NEG | POS | Partially | POS |
| | | | | | | Positive | |
| | | | | | | (PP) | |
| | Relapse | primitive | POS | POS | NEG | NEG | NEG |
| 3 | Dx | primitive | POS | POS | PP | NEG | PP |
| | Relapse | primitive | PP | PP | PP | NEG | NEG |
| 4 | Dx | primitive | NEG | NEG | Dim | NEG | Dim |
| | Relapse | primitive | NEG | NEG | POS | NEG | NEG |
| 5 | Dx | primitive | PP | NEG | PP | NEG | NEG |
| | Relapse | primitive | POS | NEG | POS | NEG | NEG |
| 6 | Dx | na | / | / | / | / | / |
| | Relapse | primitive | POS | POS | / | / | / |

In no case was a monocytic phenotype observed at relapse. For two patients that had monocytic characteristics at presentation, conversion to a more primitive phenotype was observed. The other four patients presented as primitive and remained primitive at relapse. In addition, using published RNA-seq data capturing the global transcriptome of 11 pairs of diagnostic/relapse specimens from a separate study, we found the same primitive selection at relapse from chemotherapy was observed. Together, these data indicate that relapse following conventional chemotherapy favors a primitive phenotype, and that the drive toward a monocytic phenotype observed at relapse appears to be a unique resistance mechanism to VEN+AZA.

### Example 3—Monocytic AML is Intrinsically Resistant to VEN+AZA

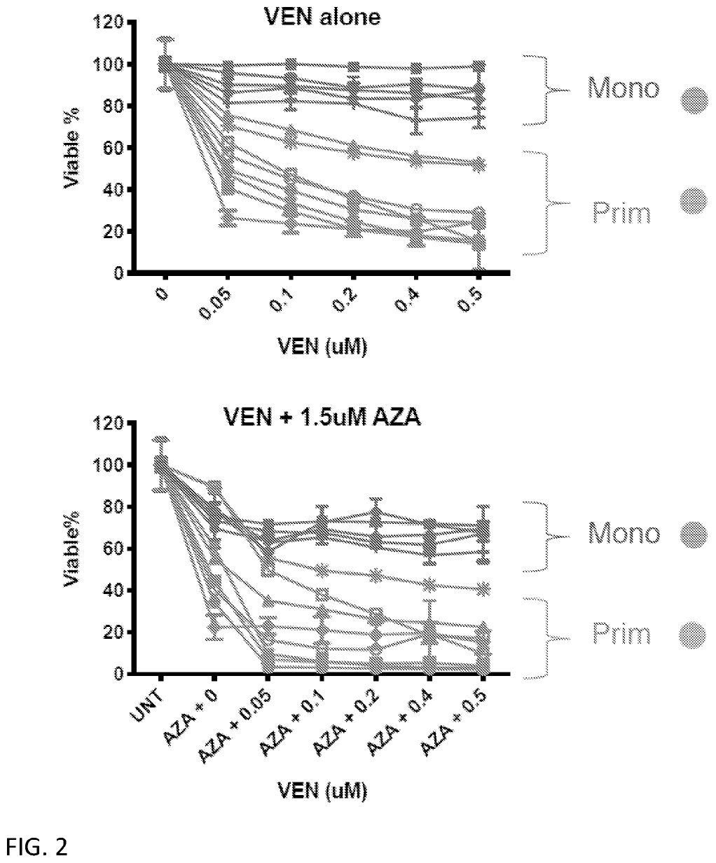

To understand if the lack of response by monocytic AML to VEN+AZA is driven by intrinsic mechanisms, VEN+AZA sensitivity was directly evaluated in vitro, where protection from extrinsic factors such as the microenvironment is minimal. For these experiments, a second cohort of AML specimens with sufficient material to allow for more robust in vitro experimentation was used (herein referred to as Research Cohort 2). Using the surface expression profile of CD117, CD11b, CD68 and CD64, the primitive or monocytic compartment was isolated for analysis (herein referred to as prim-AML or mono-AML, respectively). Prim-AML has the phenotype of CD117+/CD11b−/CD68−/CD64− and mono-AML is predominantly CD117−/CD11b+/CD68+/CD64+. The in vitro drug sensitivity of these populations is shown in FIG. 2 and closely correlates with relative responses observed for the Clinical Cohort.

The relative sensitivity of LSCs derived from primitive versus monocytic AML specimens was analyzed. Using methods described previously to enrich for LSCs, reactive oxygen species low (ROS-low) LSCs from mono- and prim-AML specimens were sorted and then treated with VEN+AZA in vitro. As shown in FIG. 2, ROS-low LSCs of the mono-AML specimens are significantly more resistant to treatment than those of the prim-AML specimens, indicating that the refractory/relapse responses seen in FAB-M5 patients can be at least partially attributed to intrinsic molecular mechanisms uniquely present in monocytic AML cells.

### Example 4—Phenotypically Defined Monocytic AMLs are Biologically Distinct from Primitive AMLs

To characterize unique molecular mechanisms present in the mono-AMLs that may be responsible for their intrinsic resistance to VEN+AZA, ROS-low LSCs from mono- and prim-AML specimens were sorted and subsequent RNA-seq analysis was performed. After removal of low abundant genes and normalization, Principle Component Analysis (PCA) of the transcriptome was performed. Mono-AMLs clustered separately from the prim-AMLs. The two groups had very distinct gene expression profiles as shown by the top 50 up and down regulated genes. Among the up-regulated genes in the mono-AML ROS-low LSCs, we identified MAFB, the master regulator of monocytic differentiation and monocytic lineage markers CD14 and LYZ. In contrast, the down-regulated genes in the mono-AML ROS-low LSCs include the classic stemness marker CD34. Furthermore, Gene Set Enrichment Analysis (GSEA) was performed and the results indicated that the prim-AMLs have enriched multiple LSC gene sets, while the mono-AMLs have enriched monocytic differentiation and AML lysosome gene sets. These results indicate that multiple transcriptional programs differ between the two AML subtypes and that the phenotypically defined monocytic AMLs reside in a distinct developmental state compared to the primitive AMLs.

### Example 5—AML at Full Monocytic Developmental Stage Loses BCL-2 but Maintains MCL-1 Dependency

To determine if differences in developmental stage underly the reduced response to VEN+AZA, expression of apoptosis family genes was analyzed. Among all genes involved in the regulation of anti-apoptosis, pro-apoptosis, effectors, and the inhibitor of apoptosis (IAP) family, the analysis revealed significant and consistent loss of BCL-2 in all five mono-AMLs, compared to the seven prim-AMLs. In contrast, MCL-1 expression was maintained at a similar level. These differences were verified at the protein level, indicating that loss of BCL-2 is a feature of AML upon monocytic differentiation.

To corroborate our analysis, TCGA-AML dataset where RNA-seq data is available for a larger number of AML patients with FAB labels was analyzed. Compared to the FAB-M0/M1/M2 AMLs, the FAB-M5 AMLs have significantly downregulated BCL-2 and upregulated MCL-1 expression. Further, loss of BCL-2 also occurs at the monocytic developmental stage during normal hematopoiesis. In particular, the expression of apoptosis family genes along the axis of long term-hematopoietic stem cells (LT-HSC), hematopoietic stem cells (HSC), common myeloid progenitors (CMP), progenitor of monocytic lineages (CFU-mono) and fully differentiated monocytes during normal human and murine hematopoiesis display consistent loss of BCL-2 and gain of MCL-1 expression at the monocytic stage in both human and murine systems. These analyses indicate that BCL-2 loss and MCL-1 maintenance/gain is a conserved inter-species biological feature in both normal and malignant monocytic developmental stages.

### Example 6—MCL-1 is Required for Hyperactive OXPHOS in Monocytic AML

The following experiments were aimed at determining whether OXPHOS in monocytic AML depends on MCL-1 over BCL-2. First, basal OXPHOS activities in the ROS-low LSCs of primitive and monocytic AMLs were measured and compared. GSEA analysis of the RNA-seq data compared against the KEGG collection demonstrated that OXPHOS was the most up-regulated gene set in monocytic AML relative to primitive AML. Consistent with this result, functional assays using the Seahorse™ XF Analyzer to directly measure oxygen consumption rate (OCR) showed basal OCR was significantly higher in the ROS-low LSCs of monocytic AML compared to their primitive counterparts.

To determine if MCL-1 was required for driving hyperactive OXPHOS activities in monocytic AML, ROS-low LSCs from multiple monocytic AMLs were sorted and subsequently cultured with VEN+AZA or MCL-1 inhibitors+AZA side by side for 3-4 hours, followed by mitochondrial respiration analysis using OCR measurements.

To inhibit MCL-1, YM155, a small molecule able to deplete the MCL-1 protein in multiple cancer types, was used. The combination of YM155 and AZA significantly depleted almost all aspects of OXPHOS in the ROS-low LSCs of monocytic AMLs, including basal respiration, maximum respiration and ATP production. This impact was selective against monocytic AML, as the same treatment had minimal impact on OXPHOS of primitive counterparts. In contrast, while VEN+AZA had the ability to significantly inhibit maximum respiration in primitive AML ROS-low LSCs, it had little to no impact on all aspects of OXPHOS in the monocytic setting.

These experiments were then repeated using another MCL-1 inhibitor, VU103, that functions as a BH3 mimetic. A similar selective inhibition of basal respiration, maximum respiration and ATP production in the ROS-low LSCs of monocytic AMLs treated with VU103+AZA was observed.

Together, the results of these experiments suggest that regulation of OXPHOS in monocytic AML switches from reliance on BCL-2 to MCL-1.

### Example 7—MCL-1 Inhibitors Selectively Target VEN+AZA Resistant Monocytic AML Cells

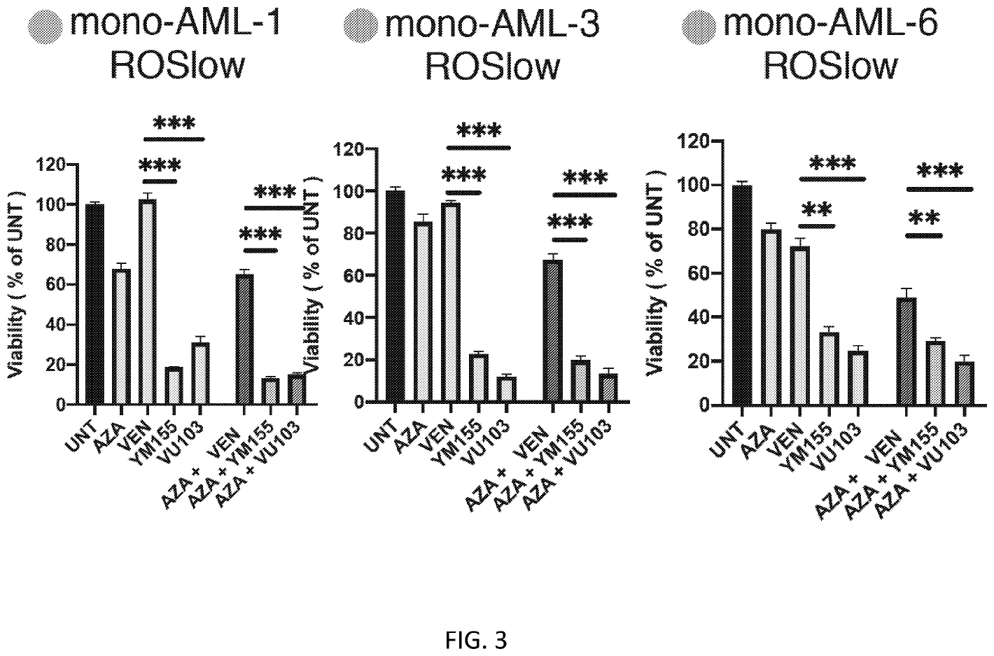

The following experiments are aimed at determining whether MCL-1 inhibition can selectively eradicate monocytic AML. Sorted ROS-low LSCs from primitive and monocytic AMLs were treated with AZA+VEN or MCL-1 inhibitors. Prim-AML specimens were sensitive to both VEN and AZA, and showed strong combined response to the two drugs together. In contrast, the same specimens were highly resistant to YM155. Conversely, mono-AML ROS-low LSCs were resistant to VEN but sensitive to YM155, resulting in 60-80% cell death when treated with the YM155+AZA combination. In a separate experiment ROS-low LSCs of monocytic AMLs were treated with both MCL-1 inhibitors YM155 and VU103 side by side with VEN. As shown in FIG. 3, the two MCL-1 inhibitors, when combined with AZA, both induced a similarly strong cell death in mono-AML ROS-low LSCs, which was significantly greater than observed with the VEN+AZA combo. Without wishing to be bound by theory, these results are consistent with the hypothesis that AML at the monocytic developmental stage loses BCL-2 dependence and switches to reliance on MCL-1.

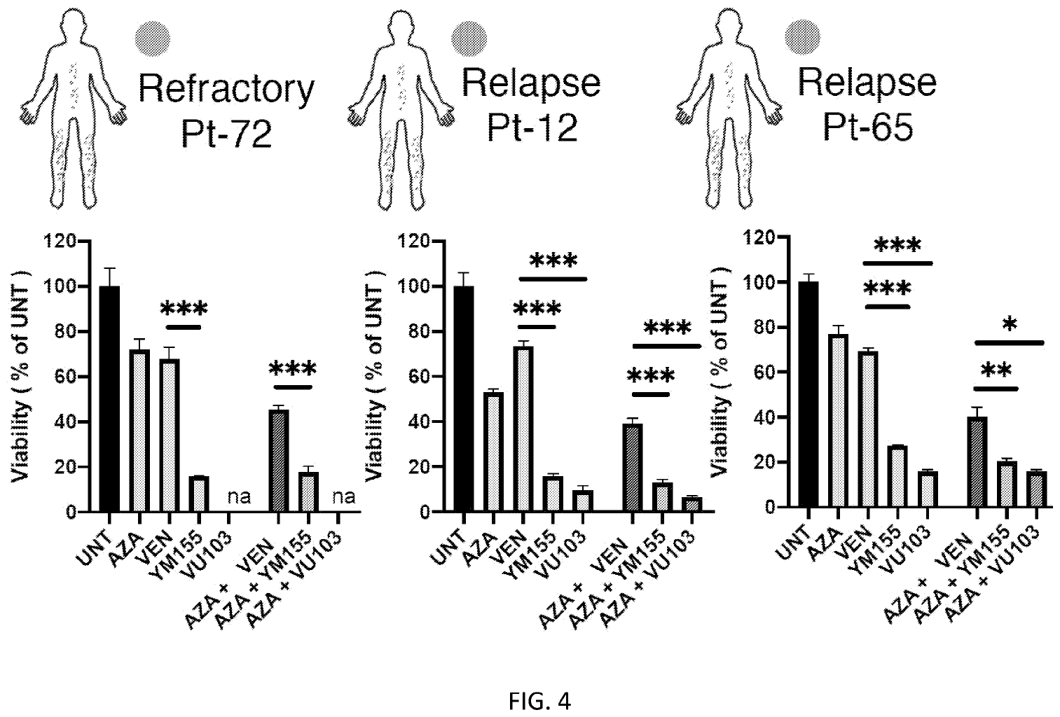

To evaluate the clinical potential of using MCL-1 inhibitors to treat monocytic patients who might be refractory to VEN+AZA or relapsed after this regimen, the same experiment was repeated in ROS-low LSCs sorted from the diagnostic bone marrow from patient Patient-72 and relapsed bone marrow from patient Patient-12 and Patient-65. As shown in FIG. 4, while all three specimens were resistant to VEN or VEN+AZA as expected, they remained sensitive to MCL-1 inhibition.

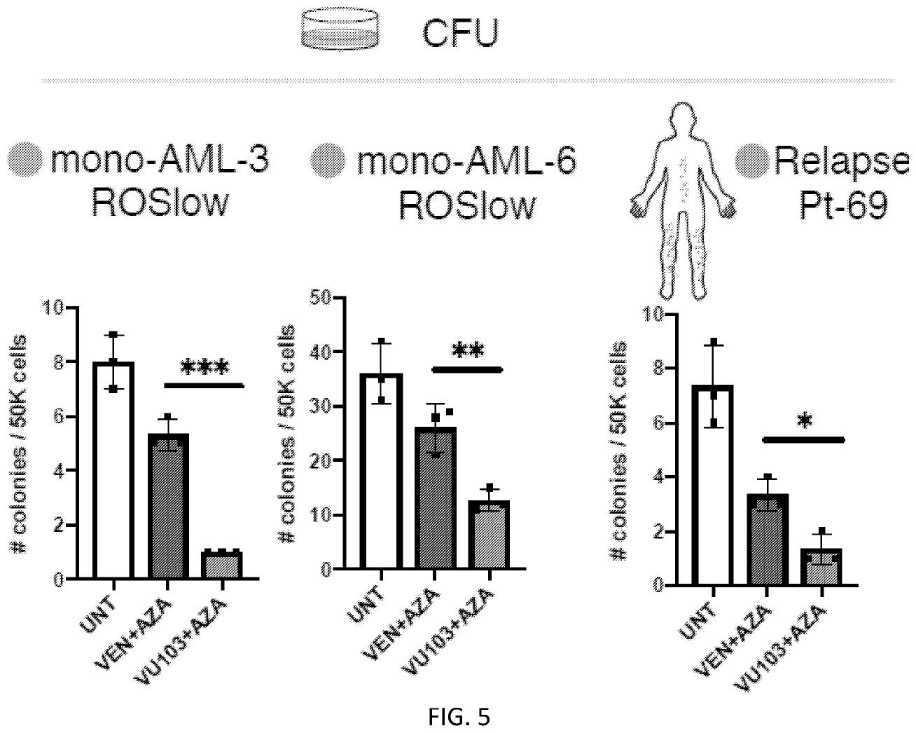

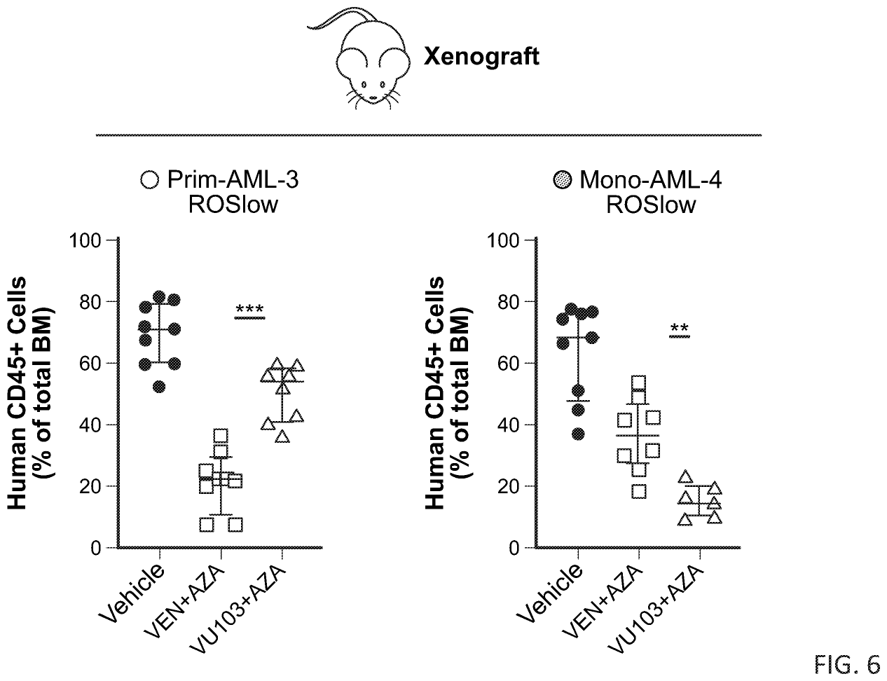

In additional experiments, two monocytic AML specimens from Research Cohort 2 and Patient-69 from Clinical Trial Cohort 1 were employed to determine whether the combination of an MCL-1 inhibitor with AZA could impair the functionally-defined stem and progenitor potential of monocytic AML. Patient-69 showed predominantly monocytic AML at relapse following treatment with VEN+AZA. As shown in FIG. 5, in all three specimens, the combination of VU103+AZA was significantly better than VEN+AZA in reducing colony-forming units. To directly assess the impact of MCL-1 inhibition on LSC potential, xenograft studies were performed using primitive and monocytic AMLs treated with VEN+AZA or MCL-1 inhibitors+AZA. As shown in FIG. 6, VEN+AZA effectively impaired the LSC engraftment potential in primitive AML, but less so in monocytic AML. The combination of AZA with MCL-1 inhibitor VU103 eradicated the LSC potential of monocytic AMLs that were otherwise resistant to the VEN+AZA therapy. Together, these results indicate that newly diagnosed or VEN+AZA refractory/relapsed monocytic AML can be effectively treated with MCL-1 inhibitors.

### Summary of Examples 1-7

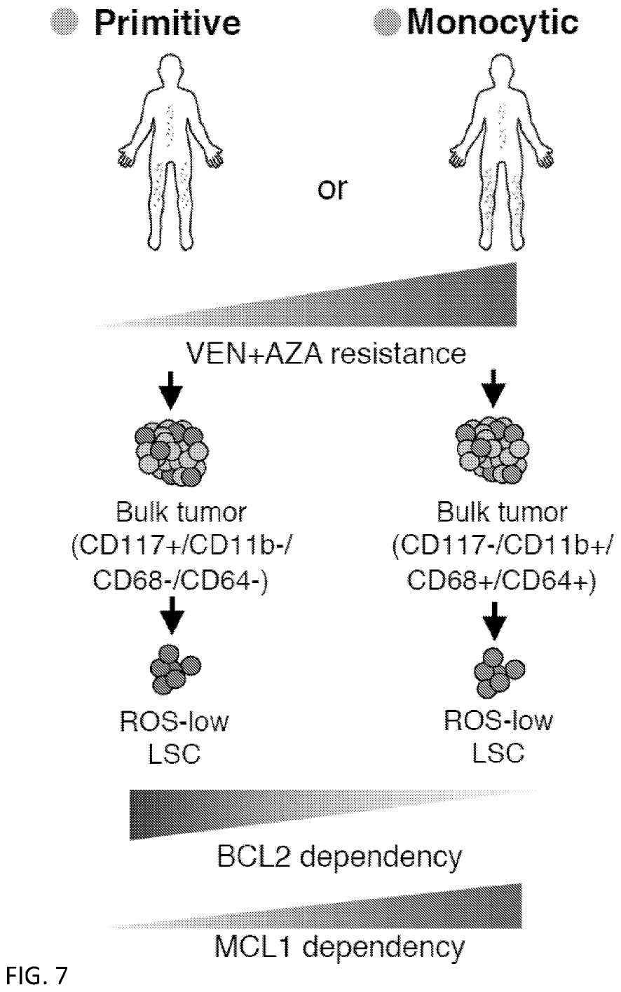

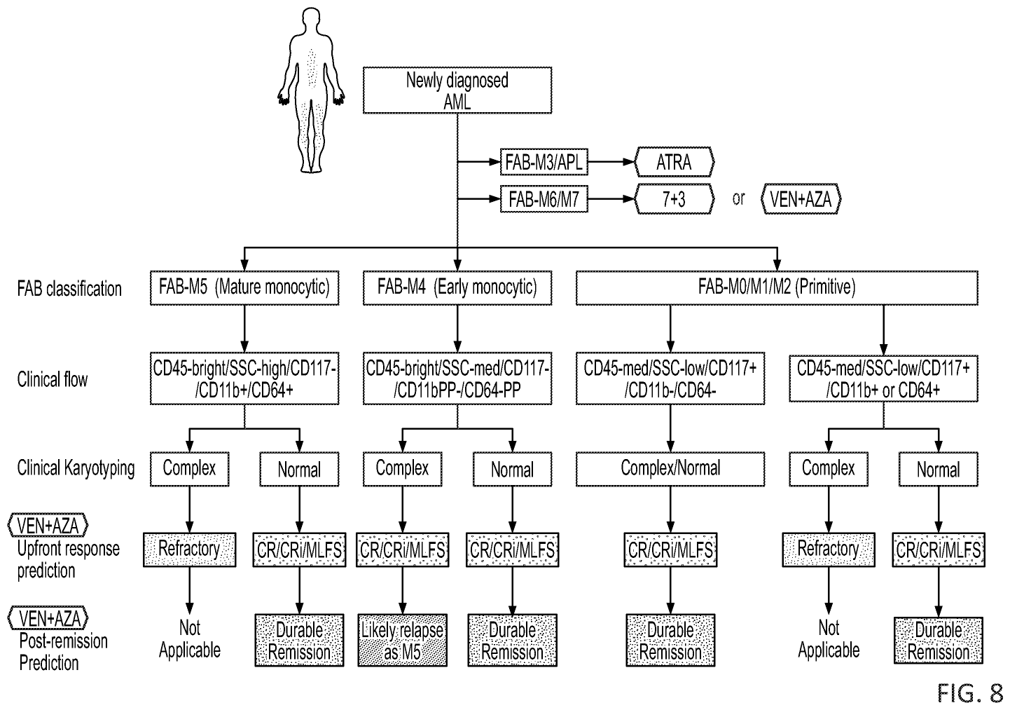

These results of the examples presented herein are surprising and demonstrate several previously unrecognized characteristics of AML. First, AML in individual patients is fluid, existing on a dynamic spectrum of differentiation. In some cases, AML is predominantly undifferentiated or monocytic, but in many instances a mixture of developmental stages is evident. Without wishing to be bound by theory, this mixture of developmental stages has implications related to responsiveness to VEN+AZA. As the role of BCL-2 is highly dependent on the relative developmental stage, with much more prevalent activity as a driver of OXPHOS in undifferentiated AML cells, the efficacy of BCL-2 targeting agents such as venetoclax varies substantially between primitive and monocytic cell types within the same patient. Moreover, without wishing to be bound by theory, MCL-1 supplants BCL-2 as a key mediator of OXPHOS and represent a key target in this AML subtype. Together, these findings indicate that utilization of venetoclax-based regimens should be tailored to the developmental stage and metabolic properties of a given AML patient. To this end, the present disclosure provides methods using morphological assessments and/or phenotypic profiling of newly diagnosed AML patients is a simple means to ascertain the likelihood of responding to venetoclax-based regimens as shown in FIG. 7. FIG. 8 is a schematic overview of a prognosis algorithm of the present disclosure for AML patients treated with VEN+AZA.

### Example 8—Response to Venetoclax and Azacitidine Therapy in Subjects with CD7+ or CD7− AML

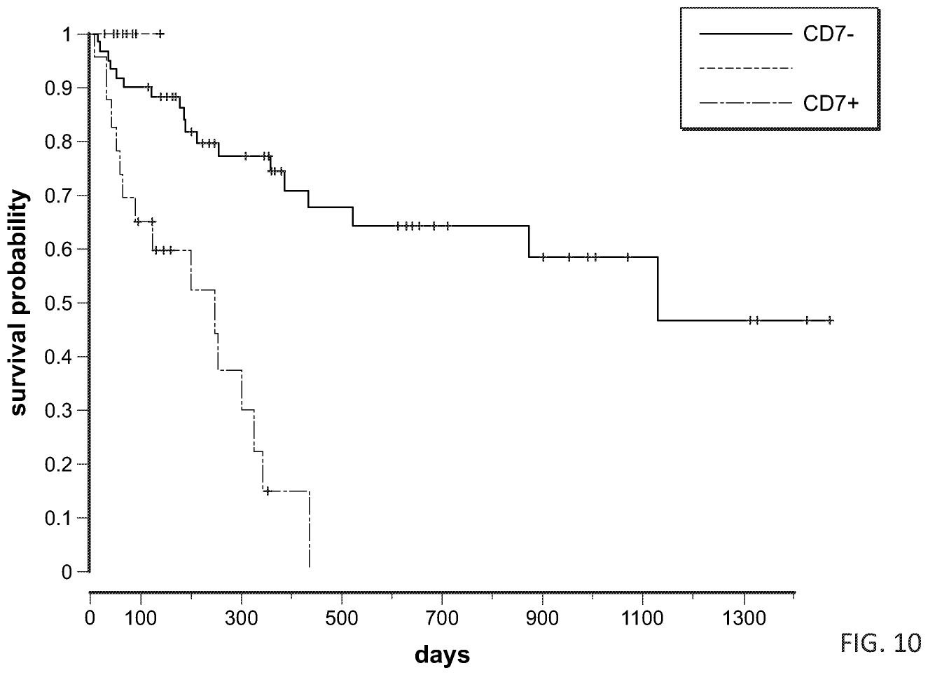

Bone marrow was collect from about 100 subjects treated with a combination of venetoclax and azacitidine. As shown in FIG. 10, analysis of cell surface markers revealed that subjects having AML cells that express CD7 (CD7+) had limited long term survival with the venetoclax/azacitidine treatment (green, or bottom line) as compared subject having AML cells that do not express CD7 (CD7−), who responded more favorably to the venetoclax/azacitidine treatment (black, or middle line). This analysis reveals that subjects that have CD7+ AML do not respond to venetoclax/azacitidine treatment and that subjects that have CD7− AML do respond to venetoclax/azacitidine treatment.

What is claimed is:

1. A method of identifying a subject having acute myeloid leukemia (AML) that will be refractory to treatment with a combination of venetoclax and azacitidine, the method comprising:
a) classifying a sample from the subject according to the French-American-British (FAB) classification system of acute myeloid leukemia;
b) determining the clinical karyotype of a sample from the subject;
c) determining side scatter intensity in a sample from the subject;
d) comparing the side scatter intensity to a first and a second predetermined cutoff value;
e) determining the expression level of each of the biomarkers selected from CD45, CD117, CD11b and CD64 in a sample from the subject;
f) comparing the expression level of each of the biomarkers to at least one corresponding predetermined cutoff value for each biomarker;
g) identifying that the subject will be refractory to treatment with a combination of venetoclax and azacitidine when:
i) the sample is classified as FAB-M5,
the clinical karyotype is complex,
the side scatter intensity is greater than or equal to the first side scatter intensity predetermined cutoff value,
the expression level of each of CD11b and CD64 is greater than or equal to its corresponding predetermined cutoff value,
the expression level of CD45 is greater than or equal to its corresponding first predetermined cutoff value, and
the expression level of CD117 is less than its corresponding predetermined cutoff value; or
ii) the sample is classified as FAB-M0, FAB-M1 or FAB-M2,
the clinical karyotype is complex,
the side scatter intensity is less than the second side scatter intensity predetermined cutoff value,
the expression level of CD11b or CD64 is greater than or equal to its corresponding predetermined cutoff value,
the expression level of CD45 is less than its corresponding first predetermined cutoff value and greater than or equal to its corresponding second predetermined cutoff value, and
the expression level of CD117 is greater than or equal to its corresponding predetermined cutoff value.

2. A method of identifying a subject having AML that will relapse after treatment with a combination of venetoclax and azacitidine, the method comprising:
a) classifying a sample from the subject according to the French-American-British (FAB) classification system of acute myeloid leukemia;
b) determining the clinical karyotype of a sample from the subject;
c) determining side scatter intensity in a sample from the subject;
d) comparing the side scatter intensity to a first and a second predetermined cutoff value;
e) determining the expression level of each of the biomarkers selected from CD45, CD117, CD11b and CD64 in a sample from the subject;
f) comparing the expression level of each of the biomarkers to a corresponding predetermined cutoff value for each biomarker;
g) identifying that the subject will be relapse after treatment with a combination of venetoclax and azacitidine when:
the sample is classified as FAB-M4,
the clinical karyotype is complex,
the side scatter intensity is greater than or equal to the first side scatter intensity predetermined cutoff value and less than the second side scatter intensity predetermined cutoff value,
the expression level of each of CD45, CD11b and CD64 is greater than or equal to its corresponding predetermined cutoff value, and
the expression level of CD117 is less than its corresponding predetermined cutoff value.

3. A method of identifying a subject having acute myeloid leukemia (AML) that will exhibit durable remission after treatment with a combination of venetoclax and azacitidine, the method comprising:
a) determining the expression level of CD7 in a sample from the subject;
b) comparing the expression level of CD7 to a predetermined cutoff value; and
c) identifying the subject will be refractory to treatment with a combination of venetoclax and azacitidine when the expression level of CD7 is less than the predetermined cutoff.

4. A method of identifying a subject having AML that will exhibit durable remission after treatment with a combination of venetoclax and azacitidine, the method comprising:
a) classifying a sample from the subject according to the French-American-British (FAB) classification system of acute myeloid leukemia;
b) determining the clinical karyotype of a sample from the subject;
c) determining side scatter intensity in a sample from the subject;
d) comparing the side scatter intensity to a first and second predetermined cutoff value;
e) determining the expression level of each of the biomarkers selected from CD45, CD117, CD11b and CD64 in a sample from the subject;
f) comparing the expression level of each of the biomarkers to at least one corresponding predetermined cutoff value for each biomarker;
g) identifying that the subject will be exhibit durable remission after treatment with a combination of venetoclax and azacitidine when:
i) the sample is classified as FAB-M5,
the clinical karyotype is normal,
the side scatter intensity is greater than or equal to the first side scatter intensity predetermined cutoff value,
the expression level of CD45 is greater than or equal to its corresponding first predetermined cutoff value,
the expression level of each of CD11b and CD64 is greater than or equal to its corresponding first predetermined cutoff value, and
the expression level of CD117 is less than its corresponding predetermined cutoff value; or
ii) the sample is classified as FAB-M4,
the clinical karyotype is normal,
the side scatter intensity is less than the first side scatter intensity predetermined cutoff value and greater than or equal to the second side scatter intensity predetermined cutoff value,
the expression level of CD45 is greater than or equal to its corresponding first predetermined cutoff value,
the expression level of each of CD11b and CD64 is greater than or equal to its corresponding second predetermined cutoff value, and
the expression level of CD117 is less than its corresponding predetermined cutoff value;
iii) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of each of CD11b and CD64 is less than its corresponding second predetermined cutoff value, and
the expression level of CD117 is greater than or equal to its corresponding predetermined cutoff value; or
iv) the sample is classified as FAB-M0, FAB-M1 or FAB-M2,
the clinical karyotype is normal or complex,
the side scatter intensity is less than the second side scatter intensity predetermined cutoff value,
the expression level of CD45 is less than its corresponding first predetermined cutoff value and greater than or equal to its corresponding second predetermined cutoff value,
the expression level of CD11b or CD64 is greater than its corresponding first predetermined cutoff value, and
the expression level of CD117 is greater than or equal to its corresponding predetermined cutoff value.

5. A method of identifying a subject having acute myeloid leukemia (AML) that will be refractory to or that will relapse from treatment with a combination of venetoclax and azacitidine, the method comprising:
a) determining the expression level of CD7 in a sample from the subject;
b) comparing the expression level of CD7 to a predetermined cutoff value; and
c) identifying that the subject will be refractory to treatment with a combination of venetoclax and azacitidine when the expression level of CD7 is equal to or greater than the predetermined cutoff.

6. The method of any of the preceding claims, wherein the sample comprises acute myeloid leukemia cells.

7. The method of any of the preceding claims, wherein the acute myeloid leukemia cells comprise acute myeloid leukemia blast cells.

8. The method of any of the preceding claims, wherein the acute myeloid leukemia cells comprise leukemia stem cells.

9. The method of any of the preceding claims, wherein the leukemia stem cells comprise reactive oxygen species-low leukemia stem cells.

10. The method of any of the preceding claims, wherein the sample is blood, a bone marrow biopsy, a bone marrow aspirate, a biopsy of a chloroma, a tissue biopsy, cerebrospinal fluid or any combination thereof.

11. The method of any of the preceding claims, wherein the sample is a bone marrow biopsy.

12. The method of any of the preceding claims, wherein the sample is a bone marrow aspirate.

13. The method of any of the preceding claims, wherein the sample is a biopsy of a chloroma.

14. The method of any of the preceding claims, wherein the sample is cerebrospinal fluid.

15. The method of any of the preceding claims, wherein the subject has been previously diagnosed with acute myeloid leukemia.

16. The method of any of the preceding claims, wherein the subject has been previously administered an initial therapy.

17. The method of any of the preceding claims, wherein the subject has not responded to the initial therapy.

18. The method of any of the preceding claims, wherein the initial therapy comprised administering to the subject a therapeutically effective amount of venetoclax in combination with a therapeutically effective amount of azacitidine.

19. The method of any of the preceding claims, wherein the initial therapy comprised anti-cancer therapy, chemotherapy, targeted drug therapy, radiation therapy, immunotherapy, stem cell transplant or any combination thereof.

20. The method of any of the preceding claims, wherein the subject is at least 18 years of age, or at least 50 years of age, or at least 60 years of age, or at least 70 years of age, or at least 80 years of age.

21. The method of any of the preceding claims, wherein determining the expression of a biomarker or CD7 comprises PCR, high-throughput sequencing, next generation sequencing, Northern Blot, reverse transcription PCR (RT-PCR), real-time PCR (qPCR), quantitative PCR, qRT-PCR, flow cytometry, mass spectrometry, microarray analysis, digital droplet PCR, Western Blot or any combination thereof.

22. The method of any of the preceding claims, wherein a complex clinical karyotype comprises the presence of at least 3 chromosomal aberrations.

23. The method of any of the preceding claims, wherein a normal clinical karyotype comprises 46 XY or 46 XX.

24. The method of any of the preceding claims, further comprising administering at least one therapeutically effective amount of at least one alternative therapy to the subject that is identified as a subject that will be refractory to or that will relapse from treatment with a combination of venetoclax and azacitidine, wherein the at least one alternative therapy does not comprise venetoclax in combination with azacitidine.

25. The method of any of the preceding claims, further comprising providing a treatment recommendation to the subject that is identified as a subject that will be refractory to or that will relapse from treatment with a combination of venetoclax and azacitidine, wherein the treatment recommendation comprises recommending the administration of at least one therapeutically effective amount of at least one alternative therapy.

26. The method of any of the preceding claims, wherein the at least one alternative therapy comprises anti-cancer therapy, chemotherapy, targeted drug therapy, radiation therapy, immunotherapy, stem cell transplant or any combination thereof.

27. The method of any of the preceding claims, further comprising administering at least one therapeutically effective amount of venetoclax in combination with a therapeutically effective amount of azacitidine to the subject that is identified as a subject that will exhibit durable remission after treatment with a combination of venetoclax and azacitidine.

28. The method of any of the preceding claims, further comprising providing a treatment recommendation to the subject that is identified as a subject that will exhibit durable remission after treatment with a combination of venetoclax and azacitidine, wherein the treatment recommendation comprises recommending the administration of at least one therapeutically effective amount of venetoclax in combination with a therapeutically effective amount of azacitidine.


SEQUENCE LISTING (12 sequences)

SEQ ID NO: 1 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 2 - RNA, 3921 nt, Homo sapiens
augaccauguauuuguggcuuaaacucuuggcauuuggcuuugccuuucuggacacagaaguauuugugacagggcaaagcccaacaccuucccccacuggauugacuacagcaaagaugcccaguguuccacuuucaagugaccccuuaccuacucacaccacugcauucucacccgcaagcaccuuugaaagagaaaaugacuucucagagaccacaacuucucuuaguccagacaauacuuccacccaaguauccccggacucuuuggauaaugcuagugcuuuuaauaccacagguguuucaucaguacagacgccucaccuucccacgcacgcagacucgcagacgcccucugcuggaacugacacgcagacauucagcggcuccgccgccaaugcaaaacucaacccuaccccaggcagcaaugcuaucucagaugucccaggagagaggaguacagccagcaccuuuccuacagacccaguuuccccauugacaaccacccucagccuugcacaccacagcucugcugccuuaccugcacgcaccuccaacaccaccaucacagcgaacaccucagaugccuaccuuaaugccucugaaacaaccacucugagcccuucuggaagcgcugucauuucaaccacaacaauagcuacuacuccaucuaagccaacaugugaugaaaaauaugcaaacaucacuguggauuacuuauauaacaaggaaacuaaauuauuuacagcaaagcuaaauguuaaugagaauguggaauguggaaacaauacuugcacaaacaaugaggugcauaaccuuacagaauguaaaaaugcgucuguuuccauaucucauaauucauguacugcuccugauaagacauuaauauuagaugugccaccagggguugaaaaguuucaguuacaugauuguacacaaguugaaaaagcagauacuacuauuuguuuaaaauggaaaaauauugaaaccuuuacuugugauacacagaauauuaccuacagauuucagugugguaauaugauauuugauaauaaagaaauuaaauuagaaaaccuugaacccgaacaugaguauaagugugacucagaaauacucuauaauaaccacaaguuuacuaacgcaaguaaaauuauuaaaacagauuuugggaguccaggagagccucagauuauuuuuuguagaagugaagcugcacaucaaggaguaauuaccuggaaucccccucaaagaucauuucauaauuuuacccucuguuauauaaaagagacagaaaaagauugccucaaucuggauaaaaaccugaucaaauaugauuugcaaaauuuaaaaccuuauacgaaauauguuuuaucauuacaugccuacaucauugcaaaagugcaacguaauggaagugcugcaaugugucauuucacaacuaaaagugcuccuccaagccaggucuggaacaugacugucuccaugacaucagauaauaguaugcaugucaaguguaggccucccagggaccguaauggcccccaugaacguuaccauuuggaaguugaagcuggaaauacucugguuagaaaugagucgcauaagaauugcgauuuccguguaaaagaucuucaauauucaacagacuacacuuuuaaggccuauuuucacaauggagacuauccuggagaacccuuuauuuuacaucauucaacaucuuauaauucuaaggcacugauagcauuucuggcauuucugauuauugugacaucaauagcccugcuuguuguucucuacaaaaucuaugaucuacauaagaaaagauccugcaauuuagaugaacagcaggagcuuguugaaagggaugaugaaaaacaacugaugaauguggagccaauccaugcagauauuuuguuggaaacuuauaagaggaagauugcugaugaaggaagacuuuuucuggcugaauuucagagcaucccgcggguguucagcaaguuuccuauaaaggaagcucgaaagcccuuuaaccagaauaaaaaccguuauguugacauucuuccuuaugauuauaaccguguugaacucucugagauaaacggagaugcagggucaaacuacauaaaugccagcuauauugaugguuucaaagaacccaggaaauacauugcugcacaaggucccagggaugaaacuguugaugauuucuggaggaugauuugggaacagaaagccacaguuauugucauggucacucgaugugaagaaggaaacaggaacaagugugcagaauacuggccgucaauggaagagggcacucgggcuuuuggagauguuguuguaaagaucaaccagcacaaaagauguccagauuacaucauucagaaauugaacauuguaaauaaaaaagaaaaagcaacuggaagagaggugacucacauucaguucaccagcuggccagaccacggggugccugaggauccucacuugcuccucaaacugagaaggagagugaaugccuucagcaauuucuucaguggucccauuguggugcacugcagugcugguguugggcgcacaggaaccuauaucggaauugaugccaugcuagaaggccuggaagccgagaacaaaguggauguuuaugguuauguugucaagcuaaggcgacagagaugccugaugguucaaguagaggcccaguacaucuugauccaucaggcuuugguggaauacaaucaguuuggagaaacagaagugaauuugucugaauuacauccauaucuacauaacaugaagaaaagggauccacccagugagccgucuccacuagaggcugaauuccagagacuuccuucauauaggagcuggaggacacagcacauuggaaaucaagaagaaaauaaaaguaaaaacaggaauucuaaugucaucccauaugacuauaacagagugccacuuaaacaugagcuggaaaugaguaaagagagugagcaugauucagaugaauccucugaugaugacagugauucagaggaaccaagcaaauacaucaaugcaucuuuuauaaugagcuacuggaaaccugaagugaugauugcugcucagggaccacugaaggagaccauuggugacuuuuggcagaugaucuuccaaagaaaagucaaaguuauuguuaugcugacagaacugaaacauggagaccaggaaaucugugcucaguacuggggagaaggaaagcaaacauauggagauauugaaguugaccugaaagacacagacaaaucuucaacuuauacccuucgugucuuugaacugagacauuccaagaggaaagacucucgaacuguguaccaguaccaauauacaaacuggaguguggagcagcuuccugcagaacccaaggaauuaaucucuaugauucaggucgucaaacaaaaacuuccccagaagaauuccucugaagggaacaagcaucacaagaguacaccucuacucauucacugcagggauggaucucagcaaacgggaauauuuugugcuuuguuaaaucucuuagaaagugcggaaacagaagagguaguggauauuuuucaagugguaaaagcucuacgcaaagcuaggccaggcaugguuuccacauucgagcaauaucaauuccuauaugacgucauugccagcaccuacccugcucagaauggacaaguaaagaaaaacaaccaucaagaagauaaaauugaauuugauaaugaaguggacaaaguaaagcaggaugcuaauuguguuaauccacuuggugccccagaaaagcucccugaagcaaaggaacaggcugaagguucugaacccacgaguggcacugaggggccagaacauucugucaaugguccugcaaguccagcuuuaaaucaagguucauag

SEQ ID NO: 3 - protein, 976 aa, Homo sapiens [sequence omitted]

SEQ ID NO: 4 - RNA, 2931 nt, Homo sapiens
augagaggcgcucgcggcgccugggauuuucucugcguucugcuccuacugcuucgcguccagacaggcucuucucaaccaucugugaguccaggggaaccgucuccaccauccauccauccaggaaaaucagacuuaauaguccgcgugggcgacgagauuaggcuguuaugcacugauccgggcuuugucaaauggacuuuugagauccuggaugaaacgaaugagaauaagcagaaugaauggaucacggaaaaggcagaagccaccaacaccggcaaauacacgugcaccaacaaacacggcuuaagcaauuccauuuauguguuuguuagagauccugccaagcuuuuccuuguugaccgcuccuuguaugggaaagaagacaacgacacgcugguccgcuguccucucacagacccagaagugaccaauuauucccucaaggggugccaggggaagccucuucccaaggacuugagguuuauuccugaccccaaggcgggcaucaugaucaaaagugugaaacgcgccuaccaucggcucugucugcauuguucuguggaccaggagggcaagucagugcugucggaaaaauucauccugaaagugaggccagccuucaaagcugugccuguugugucuguguccaaagcaagcuaucuucuuagggaaggggaagaauucacagugacgugcacaauaaaagaugugucuaguucuguguacucaacguggaaaagagaaaacagucagacuaaacuacaggagaaauauaauagcuggcaucacggugacuucaauuaugaacgucaggcaacguugacuaucaguucagcgagaguuaaugauucuggaguguucauguguuaugccaauaauacuuuuggaucagcaaaugucacaacaaccuuggaaguaguagauaaaggauucauuaauaucuuccccaugauaaacacuacaguauuuguaaacgauggagaaaauguagauuugauuguugaauaugaagcauuccccaaaccugaacaccagcaguggaucuauaugaacagaaccuucacugauaaaugggaagauuaucccaagucugagaaugaaaguaauaucagauacguaagugaacuucaucuaacgagauuaaaaggcaccgaaggaggcacuuacacauuccuaguguccaauucugacgucaaugcugccauagcauuuaauguuuaugugaauacaaaaccagaaauccugacuuacgacaggcucgugaauggcaugcuccaauguguggcagcaggauucccagagcccacaauagauugguauuuuuguccaggaacugagcagagaugcucugcuucuguacugccaguggaugugcagacacuaaacucaucugggccaccguuuggaaagcuagugguucagaguucuauagauucuagugcauucaagcacaauggcacgguugaauguaaggcuuacaacgaugugggcaagacuucugccuauuuuaacuuugcauuuaaagguaacaacaaagagcaaauccauccccacacccuguucacuccuuugcugauugguuucguaaucguagcuggcaugaugugcauuauugugaugauucugaccuacaaauauuuacagaaacccauguaugaaguacaguggaagguuguugaggagauaaauggaaacaauuauguuuacauagacccaacacaacuuccuuaugaucacaaaugggaguuucccagaaacaggcugaguuuugggaaaacccugggugcuggagcuuucgggaagguuguugaggcaacugcuuauggcuuaauuaagucagaugcggccaugacugucgcuguaaagaugcucaagccgagugcccauuugacagaacgggaagcccucaugucugaacucaaaguccugaguuaccuugguaaucacaugaauauugugaaucuacuuggagccugcaccauuggagggcccacccuggucauuacagaauauuguugcuauggugaucuuuugaauuuuuugagaagaaaacgugauucauuuauuuguucaaagcaggaagaucaugcagaagcugcacuuuauaagaaucuucugcauucaaaggagucuuccugcagcgauaguacuaaugaguacauggacaugaaaccuggaguuucuuauguugucccaaccaaggccgacaaaaggagaucugugagaauaggcucauacauagaaagagaugugacucccgccaucauggaggaugacgaguuggcccuagacuuagaagacuugcugagcuuuucuuaccagguggcaaagggcauggcuuuccucgccuccaagaauuguauucacagagacuuggcagccagaaauauccuccuuacucauggucggaucacaaagauuugugauuuuggucuagccagagacaucaagaaugauucuaauuaugugguuaaaggaaacgcucgacuaccugugaaguggauggcaccugaaagcauuuucaacuguguauacacguuugaaagugacgucugguccuaugggauuuuucuuugggagcuguucucuuuaggaagcagccccuauccuggaaugccggucgauucuaaguucuacaagaugaucaaggaaggcuuccggaugcucagcccugaacacgcaccugcugaaauguaugacauaaugaagacuugcugggaugcagauccccuaaaaagaccaacauucaagcaaauuguucagcuaauugagaagcagauuucagagagcaccaaucauauuuacuccaacuuagcaaacugcagccccaaccgacagaagcccgugguagaccauucugugcggaucaauucugucggcagcaccgcuuccuccucccagccucugcuugugcacgacgaugucuga

SEQ ID NO: 5 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 6 - RNA, 3459 nt, Homo sapiens
auggcucucagaguccuucuguuaacagccuugaccuuaugucauggguucaacuuggacacugaaaacgcaaugaccuuccaagagaacgcaaggggcuucgggcagagcgugguccagcuucagggauccaggguggugguuggagccccccaggagauaguggcugccaaccaaaggggcagccucuaccagugcgacuacagcacaggcucaugcgagcccauccgccugcagguccccguggaggccgugaacaugucccugggccugucccuggcagccaccaccagccccccucagcugcuggccuguggucccaccgugcaccagacuugcagugagaacacguaugugaaagggcucugcuuccuguuuggauccaaccuacggcagcagccccagaaguucccagaggcccuccgaggguguccucaagaggauagugacauugccuucuugauugauggcucugguagcaucaucccacaugacuuucggcggaugaaggaguuugucucaacugugauggagcaauuaaaaaaguccaaaaccuuguucucuuugaugcaguacucugaagaauuccggauucacuuuaccuucaaagaguuccagaacaacccuaacccaagaucacuggugaagccaauaacgcagcugcuugggcggacacacacggccacgggcauccgcaaagugguacgagagcuguuuaacaucaccaacggagcccgaaagaaugccuuuaagauccuaguugucaucacggauggagaaaaguuuggcgaucccuugggauaugaggaugucaucccugaggcagacagagagggagucauucgcuacgucauuggggugggagaugccuuccgcagugagaaaucccgccaagagcuuaauaccaucgcauccaagccgccucgugaucacguguuccaggugaauaacuuugaggcucugaagaccauucagaaccagcuucgggagaagaucuuugcgaucgaggguacucagacaggaaguagcagcuccuuugagcaugagaugucucaggaaggcuucagcgcugccaucaccucuaauggccccuugcugagcacuguggggagcuaugacugggcugguggagucuuucuauauacaucaaaggagaaaagcaccuucaucaacaugaccagaguggauucagacaugaaugaugcuuacuuggguuaugcugccgccaucaucuuacggaaccgggugcaaagccugguucugggggcaccucgauaucagcacaucggccugguagcgauguucaggcagaacacuggcaugugggaguccaacgcuaaugucaagggcacccagaucggcgccuacuucggggccucccucugcuccguggacguggacagcaacggcagcaccgaccugguccucaucggggccccccauuacuacgagcagacccgagggggccagguguccgugugccccuugcccagggggagggcucgguggcagugugaugcuguucucuacggggagcagggccaacccuggggccgcuuuggggcagcccuaacagugcugggggacguaaauggggacaagcugacggacguggccauuggggccccaggagaggaggacaaccggggugcuguuuaccuguuucacggaaccucaggaucuggcaucagccccucccauagccagcggauagcaggcuccaagcucucucccaggcuccaguauuuuggucagucacugagugggggccaggaccucacaauggauggacugguagaccugacuguaggagcccaggggcacgugcugcugcucaggucccagccaguacugagagucaaggcaaucauggaguucaaucccagggaaguggcaaggaauguauuugaguguaaugaucagguggugaaaggcaaggaagccggagaggucagagucugccuccauguccagaagagcacacgggaucggcuaagagaaggacagauccagaguguugugacuuaugaccuggcucuggacuccggccgcccacauucccgcgccgucuucaaugagacaaagaacagcacacgcagacagacacaggucuuggggcugacccagacuugugagacccugaaacuacaguugccgaauugcaucgaggacccagugagccccauugugcugcgccugaacuucucucuggugggaacgccauugucugcuuucgggaaccuccggccagugcuggcggaggaugcucagagacucuucacagccuuguuucccuuugagaagaauuguggcaaugacaacaucugccaggaugaccucagcaucaccuucaguuucaugagccuggacugccucguggugggugggccccgggaguucaacgugacagugacugugagaaaugauggugaggacuccuacaggacacaggucaccuucuucuucccgcuugaccuguccuaccggaagguguccacgcuccagaaccagcgcucacagcgauccuggcgccuggccugugagucugccuccuccaccgaagugucuggggccuugaagagcaccagcugcagcauaaaccaccccaucuucccggaaaacucagaggucaccuuuaauaucacguuugauguagacucuaaggcuucccuuggaaacaaacugcuccucaaggccaaugugaccagugagaacaacaugcccagaaccaacaaaaccgaauuccaacuggagcugccggugaaauaugcugucuacaugguggucaccagccauggggucuccacuaaauaucucaacuucacggccucagagaauaccagucgggucaugcagcaucaauaucaggucagcaaccuggggcagaggagccuccccaucagccugguguucuuggugcccguccggcugaaccagacugucauaugggaccgcccccaggucaccuucuccgagaaccucucgaguacgugccacaccaaggagcgcuugcccucucacuccgacuuucuggcugagcuucggaaggcccccguggugaacugcuccaucgcugucugccagagaauccagugugacaucccguucuuuggcauccaggaagaauucaaugcuacccucaaaggcaaccucucguuugacugguacaucaagaccucgcauaaccaccuccugaucgugagcacagcugagaucuuguuuaacgauuccguguucacccugcugccgggacagggggcguuugugaggucccagacggagaccaaaguggagccguucgagguccccaacccccugccgcucaucgugggcagcucugucgggggacugcugcuccuggcccucaucaccgccgcgcuguacaagcucggcuucuucaagcggcaauacaaggacaugaugagugaagggggucccccgggggccgaaccccaguag

SEQ ID NO: 7 - protein, 374 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 8 - RNA, 1125 nt, Homo sapiens
augugguucuugacaacucugcuccuuuggguuccaguugaugggcaaguggacaccacaaaggcagugaucacuuugcagccuccaugggucagcguguuccaagaggaaaccguaaccuugcacugugaggugcuccaucugccugggagcagcucuacacagugguuucucaauggcacagccacucagaccucgacccccagcuacagaaucaccucugccagugucaaugacaguggugaauacaggugccagagaggucucucagggcgaagugaccccauacagcuggaaauccacagaggcuggcuacuacugcaggucuccagcagagucuucacggaaggagaaccucuggccuugaggugucaugcguggaaggauaagcugguguacaaugugcuuuacuaucgaaauggcaaagccuuuaaguuuuuccacuggaauucuaaccucaccauucugaaaaccaacauaagucacaauggcaccuaccauugcucaggcaugggaaagcaucgcuacacaucagcaggaauaucugucacugugaaagagcuauuuccagcuccagugcugaaugcaucugugacauccccacuccuggaggggaaucuggucacccugagcugugaaacaaaguugcucuugcagaggccugguuugcagcuuuacuucuccuucuacaugggcagcaagacccugcgaggcaggaacacauccucugaauaccaaauacuaacugcuagaagagaagacucuggguuauacuggugcgaggcugccacagaggauggaaauguccuuaagcgcagcccugaguuggagcuucaagugcuuggccuccaguuaccaacuccugucugguuucauguccuuuucuaucuggcagugggaauaauguuuuuagugaacacuguucucugggugacaauacguaaagaacugaaaagaaagaaaaagugggauuuagaaaucucuuuggauucuggucaugagaagaagguaauuuccagccuucaagaagacagacauuuagaagaagagcugaaaugucaggaacaaaaagaagaacagcugcaggaaggggugcaccggaaggagccccagggggccacguag

SEQ ID NO: 9 - protein, 354 aa, Homo sapiens
MRLAVLFSGALLGLLAAQGTGNDCPHKKSATLLPSFTVTPTVTESTGTTSHRTTKSHKTTTHRTTTTGTTSHGPTTATHNPTTTSHGNVTVHPTSNSTATSQGPSTATHSPATTSHGNATVHPTSNSTATSPGFTSSAHPEPPPPSPSPSPTSKETIGDYTWTNGSQPCVHLQAQIQIRVMYTTQGGGEAWGISVLNPNKTKVQGSCEGAHPHLLLSFPYGHLSFGFMQDLQQKVVYLSYMAVEYNVSFPHAAQWTFSAQNASLRDLQAPLGQSFSCSNSSIILSPAVHLDLLSLRLQAAQLPHTGVFGQSFSCPSDRSILLPLIIGLILLGLLALVLIAFCIIRRRPSAYQAL

SEQ ID NO: 10 - RNA, 984 nt, Homo sapiens
augaggcuggcugugcuuuucucgggggcccugcuggggcuacuggcagagagcacuggaacaaccagccacaggacuaccaagagccacaaaaccaccacucacaggacaaccaccacaggcaccaccagccacggacccacgacugccacucacaaccccaccaccaccagccauggaaacgucacaguucauccaacaagcaauagcacugccaccagccagggacccucaacugccacucacaguccugccaccacuagucauggaaaugccacgguucauccaacaagcaacagcacugccaccagcccaggauucaccaguucugcccacccagaaccaccuccacccucuccgaguccuagcccaaccuccaaggagaccauuggagacuacacguggaccaaugguucccagcccuguguccaccuccaagcccagauucagauucgagucauguacacaacccaggguggaggagaggccuggggcaucucuguacugaaccccaacaaaaccaagguccagggaagcugugagggugcccauccccaccugcuucucucauuccccuauggacaccucagcuuuggauucaugcaggaccuccagcagaagguugucuaccugagcuacauggcgguggaguacaauguguccuucccccacgcagcacaguggacauucucggcucagaaugcaucccuucgagaucuccaagcaccccuggggcagagcuucaguugcagcaacucgagcaucauucuuucaccagcuguccaccucgaccugcucucccugaggcuccaggcugcucagcugccccacacaggggucuuugggcaaaguuucuccugccccagugaccgguccaucuugcugccucucaucaucggccugauccuucuuggccuccucgcccuggugcuuauugcuuucugcaucauccggagacgcccauccgccuaccaggcccucuga

SEQ ID NO: 11 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 12 - RNA, 1284 nt, Homo sapiens
cucucugagcucugagcgccugcggucuccugugugcugcucucugugggguccuguagacccagagaggcucagcugcacucgcccggcugggagagcuggguguggggaacauggccgggccuccgaggcuccugcugcugccccugcuucuggcgcuggcucgcggccugccuggggcccuggcugcccaagaggugcagcagucuccccacugcacgacuguccccgugggagccuccgucaacaucaccugcuccaccagcgggggccugcgugggaucuaccugaggcagcucgggccacagccccaagacaucauuuacuacgaggacgggguggugcccacuacggacagacgguuccggggccgcaucgacuucucagggucccaggacaaccugacuaucaccaugcaccgccugcagcugucggacacuggcaccuacaccugccaggccaucacggaggucaaugucuacggcuccggcacccugguccuggugacagaggaacagucccaaggauggcacagaugcucggacgccccaccaagggccucugcccucccugccccaccgacaggcuccgcccucccugacccgcagacagccucugcccucccugacccgccagcagccucugcccucccugcggcccuggcggugaucuccuuccuccucgggcugggccuggggguggcgugugugcuggcgaggacacagauaaagaaacugugcucguggcgggauaagaauucggcggcaugugugguguacgaggacaugucgcacagccgcugcaacacgcuguccucccccaaccaguaccagugacccagugggccccugcacgucccgccugugguccccccagcaccuucccugccccaccaugccccccacccugccacaccccucacccugcuguccucccacggcugcagcagaguuugaagggcccagccgugcccagcuccaagcagacacacaggcaguggccaggccccacggugcuucucaguggacaaugaugccuccuccgggaagccuucccugcccagcccacgccgccaccgggaggaagccugacuguccuuuggcugcaucucccgaccauggccaaggagggcuuuucugugggaugggccugggcacgcggcccucuccugucagugccggcccacccaccagcaggcccccaacccccaggcagcccggcagaggacgggaggagaccagucccccacccagccguaccagaaauaaaggcuucugugcuucc